(19) United States
(12) Patent Application Publication
(10) Pub. No.: US 2022/0297116 A1
(43) Pub. Date: Sep. 22, 2022

(54) DETECTION OF MIRNA USING LNA PROBES AND A MEMBRANE FOR COMPLEX MIGRATION

(71) Applicant: Ohio State Innovation Foundation, Columbus, OH (US)

(72) Inventors: Peixuan Guo, Dublin, OH (US); Hongran Yin, Dublin, OH (US); Mario Vieweger, Columbus, OH (US)

(21) Appl. No.: 17/625,248

(22) PCT Filed: Jul. 8, 2020
(86) PCT No.: PCT/US2020/041262

§ 371 Date: Jan. 6, 2022

Related U.S. Application Data
(60) Provisional application No. 62/871,295, filed on Jul. 8, 2019.

(51) Int. Cl. B01L 3/00; C12Q 1/6834; C12Q 1/70; C12Q 1/6886

(52) U.S. Cl. CPC B01L 3/5023; C12Q 1/6834; C12Q 1/701; C12Q 1/6886; B01L 2300/0636; B01L 2400/0406; B01L 2300/0825

(57) ABSTRACT

Disclosed herein is a system for detecting the presence of a target molecule, such as a target single stranded nucleic acid or polypeptide, in a fluid sample. The system can involve a lateral flow device that includes a porous lateral flow test strip, a solvent reservoir, and an immobilized oligonucleotide that selectively binds to a first portion of the target molecule affixed to the lateral flow test strip at a detection spot or line. The system can further involve a marker oligonucleotide selectively binds a second portion of the target molecule conjugated to a detection reagent. The system can further involve a control oligonucleotide complementary to a portion of the marker oligonucleotide affixed to the lateral flow test strip at a control spot or line. The system can also involve a solvent configured to drag a sample through the lateral flow test strip by capillary forces.

### CROSS-REFERENCE TO RELATED APPLICATIONS

This application claims benefit of U.S. Provisional Application No. 62/871,295, filed Jul. 8, 2019, which is hereby incorporated herein by reference in its entirety.

### STATEMENT REGARDING FEDERALLY SPONSORED RESEARCH OR DEVELOPMENT

This invention was made with Government Support under Grant No. U01CA207946 and R01EB012135 awarded by the National Institutes of Health. The Government has certain rights in the invention.

### SEQUENCE LISTING

This application contains a sequence listing filed in electronic form as an ASCII.txt file entitled “321501_2120_Sequence_Listing_ST25” created on Jun. 16, 2020.

The content of the sequence listing is incorporated herein in its entirety.

### BACKGROUND

When the first human genome was completely sequenced decades ago, it was found that only 1.5% of the human genome codes for protein. A large portion of the remaining 98.5% was proposed to be “Junk DNA”. However, more and more evidence revealed that a substantial part of the so-called “Junk DNA” encodes small non-coding RNA. Recently, many long non-coding RNAs were identified with the advancement of technology, making it possible to characterize long RNA molecules. It has been predicted that the third milestone in drug development will be RNA drugs, either RNA itself as drugs or chemicals/ligands that target RNA, following the first milestone of chemical drugs and the second milestone of protein drugs including antibodies, enzymes, hormones or chemicals/ligands that target proteins.

Recent evidence has revealed that miRNAs plays a major role in the regulation of cellular function. miRNA in particular has been observed in RNA silencing and post-transcriptional regulation of gene expression. Some miRNAs are observed in the extracellular environment such as biological fluids and cell culture media. Differences in the expression rate of miRNAs in healthy and diseased such as cancer cells make these circulating miRNAs or extracellular miRNAs promising biomarkers for disease detection. Routine analysis of miRNAs include qPCR, microarrays, and high-throughput sequencing; techniques that are expensive, labor intensive, and not available in a typical doctor's office.

### SUMMARY

Disclosed herein is a system for detecting the presence of a target molecule, such as a target single stranded nucleic acid or polypeptide, in a fluid sample. The system can involve a lateral flow device that includes a porous lateral flow test strip, a solvent reservoir, and an immobilized oligonucleotide that selectively binds to a first portion of the target molecule affixed to the lateral flow test strip at a detection spot or line. The system can further involve a marker oligonucleotide selectively binds a second portion of the target molecule conjugated to a detection reagent. The system can further involve a control oligonucleotide complementary to a portion of the marker oligonucleotide affixed to the lateral flow test strip at a control spot or line. The system can also involve a solvent configured to drag a sample through the lateral flow test strip by capillary forces.

Also disclosed is a method for detecting the presence of a target molecule in a fluid sample that involves contacting the fluid sample with a marker oligonucleotide that selectively binds to a first portion of the target molecule conjugated to a detection reagent under conditions suitable for the marker that selectively binds to hybridize to the target molecule, and administering the sample after this step to the disclosed lateral flow device under conditions suitable for the solvent to pull the sample through the lateral flow test strip by capillary forces. The method can then involve assaying the lateral flow test strip for the detection reagent at the detection spot or line.

In some embodiments, the lateral flow test strip comprises a nitrocellulose, cellulose acetate, polyvinylidene difluoride (PVDF), polycarbonate, or glass fiber membrane.

In some embodiments, the target molecule is a single stranded nucleic acid molecule. In these embodiments, the immobilization oligonucleotide can be complementary to the first portion of the target nucleic acid, and the marker oligonucleotide can be complementary to the second portion of the target molecule. For example, in some embodiments, the target single stranded nucleic acid comprises a mRNA, ncRNA, sRNA, piRNA, miRNA, tRAN, rRNA, siRNA, lncRNA, snoRNAs, snRNAs, exRNAs, or scaRNAs. In some embodiments, the target single stranded nucleic acid comprises Xist or HOTAIR noncoding RNA.

In some embodiments, the target molecule is a polypeptide. In these embodiments, the immobilization oligonucleotide can be a nucleic acid (e.g. DNA) aptamer, and the marker oligonucleotide can be a nucleic acid (e.g. DNA) aptamer. In some embodiments, the immobilization oligonucleotide is replaced with a protein binding agent, such as an antibody.

In some embodiments, the disclosed nucleic acid aptamers comprise one or more locked nucleic acids, 2′-Fluoro RNA, 2′-OMethyl RNA, RNA, DNA, or Phosphorothioate-DNA. In some embodiments, the disclosed nucleic acid aptamers comprises one or more locked nucleic acids, locked nucleic acids, 2′-Fluoro RNA, 2′-OMethyl RNA, RNA, DNA, or Phosphorothioate-DNA. In some embodiments, the control oligonucleotide comprises one or more locked nucleic acids, DNA, or Phosphorothioate-DNA.

In some embodiments, the target molecule is a viral protein. For example, in some embodiments, the viral protein is a SARS-CoV-2 N protein or S protein. In some embodiments, the viral protein is HIV gp120 or gp41 protein.

In some embodiments the target molecule is a cancer biomarker. Therefore, also disclosed is a method of diagnosing or prognosing a cancer in a subject that involves assaying a bodily fluid from the subject using the disclosed device, system, and/or methods using marker and immobilization oligonucleotides that are complementary to a portion of a target nucleic acid that is a biomarker of cancer disease or progression.

In some embodiments, the fluid sample comprises serum, plasma, urine, semen, or saliva.

In some embodiments, the immobilization oligonucleotide is conjugated to an immobilization agent, wherein the immobilization oligonucleotide is affixed to the lateral flow test strip by the immobilization agent. Likewise, the control oligonucleotide can be conjugated to an immobilization agent, wherein the control oligonucleotide is affixed to the lateral flow test strip by the immobilization agent. In some embodiments, the immobilization agent is biotin. In some embodiments, the immobilization agent is a chemical crosslinker. In some embodiments, the immobilization agent is a lipid, wherein the lipid immobilizes the immobilization oligonucleotide to the lateral flow test strip by hydrophobic force such as cholesterol, or chemical immobilization or crosslinking.

In some embodiments, the detection agent comprises a fluorescence molecule, a dyed microsphere, quantum dot, fluorescence quencher molecule, intercalating fluorescence dye, a gold nanoparticle, or an iron oxide nanoparticle.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of an embodiment of a device, system, and method for detecting miRNA for cancer diagnosis.

FIGS. 2A and 2B show hybridization study of fluorescent-LNA and miRNA21.

FIGS. 3A and 3B show flow of fluorescent-RNA on the nitrocellulose membrane.

FIGS. 4A to 4C show cholesterol helps RNA anchor to nitrocellulose membrane.

FIGS. 5A to 5D show anchor efficiency of cholesterol at 3WJ different locations.

FIGS. 6A and 6B show effects of different amounts of cholesterol on anchoring.

FIGS. 7A to 7C show miR21+LNA2-AF647 bind with LNA1 and LNA3 on NC membrane. Due to lack of negative controls, specific binding cannot be determined.

FIGS. 8A and 8B show LNA2-AF647 non-specific bind with LNA1 on NC membrane.

FIGS. 9A to 9C show whether cholesterol affects LNA2-AF647 non-specific bind with LNA1 on NC membrane.

FIGS. 10A to 10D show a change from LNA2 to 2′F RNA to resolve non-specific binding.

FIG. 11 shows 2′F anti21-AF647 specific bind with miR21 and no bind with miR21-S.

FIG. 12 shows electrophoresis detection of colloidal gold label LNA2.

FIGS. 13A to 13D show the binding ability of labelling different concentration of colloidal gold-LNA2 to LNA3 on NC membrane.

FIG. 14 shows the binding ability of colloidal gold-LNA2 to anchored different concentration of LNA3 on NC membrane.

FIG. 15 shows a two LNA based probe sandwich method for cancer diagnosis.

FIG. 16 shows a hybridization study of LNA-RNA.

FIG. 17 shows a hybridization study of LNA-RNA.

FIGS. 18A to 18C show lateral flow of RNA samples on new NC membrane.

FIGS. 19A to 19C show lateral flow of RNA samples on new NC membrane.

FIG. 20 shows LNA-A647 hybridization

FIG. 21 shows LNA-A647 lateral flow on NC membrane.

FIG. 22 shows an example lateral flow assay development device.

FIG. 23 shows an example of a lateral flow assay development workflow.

FIG. 24 shows RNA anchor on NC membrane by RNA lipid.

FIG. 25 shows conjugate of AF647 to LNA2 tested for lateral flow on NC membrane.

FIGS. 26A and 26B show lateral flow of NC membranes immobilized with LNA1-chol (FIG. 26A) or LNA3-chol (FIG. 26B) for miR21 diagnosis.

FIGS. 27A and 27B show lateral flow of LNA2-AF647 (FIG. 27A) or 3WJ-b-Alexa647 (FIG. 27B) on LNA1-NC membrane for miR21 diagnosis

FIG. 28 shows lateral flow of LNA2-AF647 with nothing, LNA3-chol, LNA1-chol, or LNA1-chol+miR21.

FIG. 29A shows false positive signal in lateral flow test using LNA2-AF647.

FIG. 29B shows no signal using another RNA strand with AF647.

FIGS. 30A to 30C show false positive signal is not from cholesterol.

FIGS. 31A to 31C show 2′F modified anti-miR21-2-AF647 can also bind to miR21 efficiently in hybridization study.

FIGS. 32A to 32D show a repeat of lateral flow test showing 2′F modification can eliminate the non-specific reaction with LNA1.

FIG. 33 shows hybridization of miR21 with an extended 2′F RNA with miR21.

FIG. 34 shows illustration of the design of fast diagnosis device for COVID-19 to detect SARS-CoV-2 using nitrocellulose membrane.

FIG. 35A shows hybridization of miR21 of RNA probes assayed by 20% Native PAGE (Red: Alexa647 channel, Green: EtBr channel). FIG. 35B shows detection of miR21 on NC anchored with fixing probe (left: miR21+marker probe, right: marker probe; arrow shows the detection spot). FIG. 35C shows gold conjugation to RNA assayed by 1% Agarose gel.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “lateral flow” refers to a system where a sample suspected of containing a target nucleic acid is placed on a test strip comprising a chromatographic material and the sample is wicked laterally through of the test strip by capillary action and binds to various reagents in the strip.

Locked nucleic acid or “LNA” means a modified RNA nucleotide in which the ribose moiety is modified with an extra bridge connecting the 2′ oxygen and 4′ carbon. Locked nucleic acids can increase complex stability approximately tenfold and can alter the hybridization temperature of a nucleic acid to a probe.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “oligonucleotide probe” refers to a nucleic acid which has a sequence complementary to a portion of the target nucleic acid and which is further coupled to a binding partner. The oligonucleotide probe may either be reversibly bound to the sample receiving zone of a test strip, and/or may be used to label the target nucleic acid prior to introduction to the lateral flow system as described herein (in the latter case the oligonucleotide probe is also referred to as a “primer”).

The terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-S′. Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the invention. The term “substantially complementary” refers to any probe that can hybridize to either or both strands of the target nucleic acid sequence under conditions of high stringency as described below or, preferably, in polymerase reaction buffer, heated to about 956° C. and then cooled to about room temperature (e.g., 250° C.±3° C.).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “specifically binds”, as used herein refers to a binding reaction which is determinative of the presence of the target molecule in a heterogeneous population of like molecules. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-CI, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The term “sample from a subject” refers to a tissue (e.g., tissue biopsy), organ, cell (including a cell maintained in culture), cell lysate (or lysate fraction), biomolecule derived from a cell or cellular material (e.g. a polypeptide or nucleic acid), or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

### Lateral Flow Device

Disclosed herein is a lateral flow assay device for detecting the presence or absence of a target molecule in a fluid sample, said device comprising a test strip having a first and second end and comprising a sample receiving zone (sample pad) at or near said first end for receiving a sample, a capture zone in lateral flow contact with the sample receiving zone, and an absorbent zone (absorbant pad) positioned at or near the second end of said test strip, said absorbent zone being in lateral flow contact with the capture zone.

In some embodiments, the sample receiving zone is a porous material containing marker oligonucleotide probes, wherein the marker oligonucleotide probes specifically bind to a first portion of the target molecule to form a target complex. In some embodiments, the marker oligonucleotide is conjugated to a detectable moiety.

In some embodiments, the capture zone contains in a portion thereof a immobilization oligonucleotide immobilized thereto which specifically binds a second portion of the target molecule. In some embodiments, the target complex is captured by the immobilization oligonucleotide in the capture zone.

In some embodiments, the test strip further comprises a control zone in lateral flow contact with the sample receiving zone and absorbent zone, wherein the control zone contains a control probe immobilized thereto that specifically hybridizes to unbound marker oligonucleotide probes.

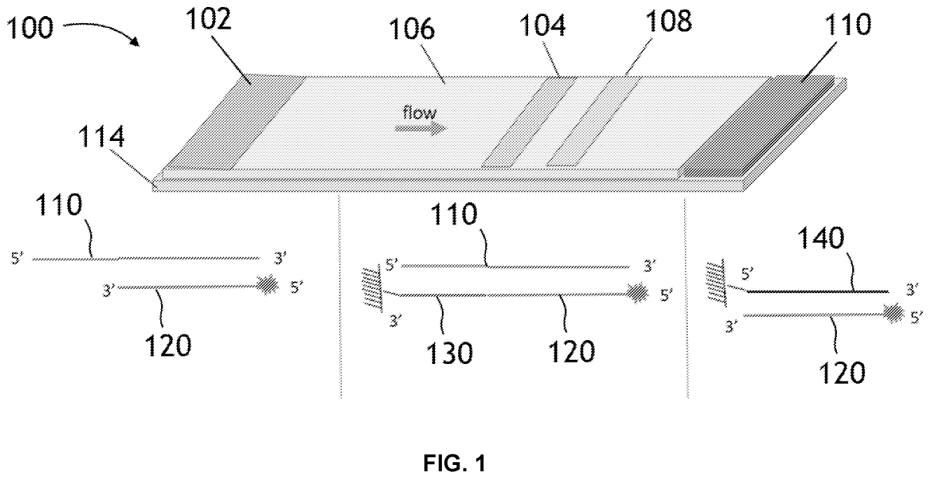

One embodiment of a lateral flow device (e.g. test strip) 100 of this invention is shown in FIG. 1. In the embodiment shown in FIG. 1, the lateral flow membrane 106 extends the length of the test strip, and the sample receiving zone material 102 is affixed to the lateral flow membrane 106. The sample receiving zone 102 serves to receive a fluid sample which may contain the target molecule 110 and to begin the flow of the sample along the test strip. The sample receiving zone 102 is prepared from a natural or synthetic porous or macroporous material which is capable of conducting lateral flow of the fluid sample. A porous or macroporous material suitable for purposes of a lateral flow device generally has a pore size greater than 12 μm. Examples of porous materials include, but are not limited to, glass, cotton, cellulose, polyester, rayon, nylon, polyethersulfone, and polyethylene.

The sample receiving zone 102 material must be a material that does not irreversibly bind nucleic acids (i.e., the oligonucleotide probes and the target nucleic acid). Rather the sample receiving zone 102 material must sufficiently retain the oligonucleotide probe on or within the sample receiving zone in an anhydrous form prior to use of the lateral flow device, but must also release the oligonucleotide probe upon contact with the fluid sample and also allow lateral flow of the target nucleic acid. The solution used to prepare the fluid sample also plays a role in rehydrating and thus releasing the oligonucleotide probes from the sample zone receiving material, as discussed below.

In one embodiment, the sample receiving zone 102 material contains anhydrous forms of one or more marker oligonucleotide probes 120 that selectively bind to a first region of the target molecule 110.

The marker oligonucleotide probes may be reversibly bound to the sample receiving zone 102 material directly by vacuum transfer, or by other well-known methods such as drying and desiccation. In this embodiment, the oligonucleotide probe functions to label the target nucleic acid with a binding partner by hybridizing with it as it passes through the sample receiving zone of the test strip.

The lateral flow membrane 106 comprises a microporous material which is capable of conducting lateral flow and is in lateral flow contact with the labeling zone material. Materials suitable for the capture zone membrane include, but are not limited to, microporous materials having a pore size from about 0.05 μm to 12 μm, such as nitrocellulose, polyethersulfone, polyvinylidine fluoride, nylon, charge-modified nylon, and polytetrafluoroethylene.

The capture zone 104 comprises immobilization oligonucleotides 130 that specifically binds a second portion of the target molecule 110. The arrangement of the first capture moiety in the capture zone may be, for example, in the form of a dot, line, curve, band, cross, or combinations thereof.

In some embodiments, the assay further contains a control probe 140 immobilized in the capture zone 104 or in a separate control zone 108 that specifically hybridizes to unbound marker oligonucleotide probes 120. The arrangement of the control probe 140 in the capture zone 104 or control zone 108 may be in the form of a dot, line, curve, band, cross, or combinations thereof.

In one embodiment, as shown in FIG. 1, the control zone 108 are in a region that is separate from the capture zone 104. Alternatively, the immobilization oligonucleotides 130 and control probes 140 are contained within the capture zone 104. Therefore, in some embodiments the marker oligonucleotides 120 and control probes 140 contain detection moieties of different colors. The control region 108 is helpful in that appearance of a color in the control region 108 signals the time at which the test result can be read, even for a negative result. Thus, when the expected color appears in the control region 108, the presence or absence of a color in the capture zone 104 can be noted.

Methods of immobilizing the capture moieties to the membrane are well known in the art. In general, the test and control capture moieties can be dispensed onto the membrane as spaced parallel lines (i.e., to form regions 104 and 108, respectively) with a linear reagent dispensing system using a solution of the test capture moiety diluted with a suitable buffer and a solution of the control capture moiety diluted with a suitable buffer. After air drying for a suitable period of time, the membrane is blocked with an appropriate buffer and stored in a desiccator until assembly of the test strip.

The absorbent pad or zone 110 is an absorbent material that is placed in lateral flow contact with the capture zone at the distal end of the test strip. In the embodiment shown in FIG. 1, the absorbent pad 110 is affixed to the capture zone membrane 106 on the same side of the membrane as the sample receiving zone and the labeling zone. The absorbent pad 110 helps to draw a test sample from the sample receiving zone to the distal end of the test strip by capillary action. Examples of materials suitable for use as an absorbent pad include any absorbent material, include, but are not limited to, nitrocellulose, cellulose esters, glass (e.g., borosilicate glass fiber), polyethersulfone, and cotton.

In the embodiment illustrated in FIG. 1, the lateral flow membrane 106 is affixed to a rigid or semi-rigid support 114, which provides structural support to the test strip. The support can be made of any suitable rigid or semi-rigid material, such as poly(vinyl chloride), polypropylene, polyester, and polystyrene. The lateral flow membrane 106 may be affixed to the support 114 by any suitable adhesive means such as with a double-sided adhesive tape. Alternatively, the support 114 may be a pressure sensitive adhesive laminate, e.g., a polyester support having an acrylic pressure sensitive adhesive on one side that is optionally covered with a release liner prior to application to the membrane.

The solution used to prepare the fluid sample contains reagents that rehydrate the oligonucleotide probes, thereby releasing the probes from the test strip. For example, the probes can be released form the material simply by rehydrating with water. It is known in the art that additional “release agents” such as surfactants, gelatin (e.g., fish skin gelatin), polymers (e.g., polyvinyl pyrrolidone), Tween 20, and sugars (e.g., sucrose or sorbitol) can facilitate the release of the probes. Thus, when the fluid sample is applied to the sample receiving zone, the target nucleic acid in the sample specifically hybridizes with the first and second oligonucleotide probes to form a complex comprising first and second binding partners (A) and (B). The target nucleic acid/visible moiety complex continues flowing with the fluid sample along the test strip 100 by capillary action in the direction of the labeling zone 104.

The disclosed assays can be performed under high or low stringency conditions. The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together. Those skilled in the art know that numerous equivalent conditions can be employed to comprise low stringency conditions. Hybridization under stringent conditions requires a perfect or near perfect sequence match. Hybridization under relaxed conditions allows hybridization between sequences with less than 100% identity. Greater stringency can be achieved by reducing the salt concentration or increasing the temperature of the hybridization.

In some embodiments the disclosed lateral flow device includes a heating sheet, and the assay can be performed at temperatures above room temperature. In some embodiments, the assay is conducted at a temperature between about 25 and 95° C. Performing lateral flow assays at high temperatures is useful for a number of applications, including forensic medicine, and for determining Watson-Crick complementarity between nucleic acid strands.

In some embodiments, the disclosed assay is a complete, one-step, ready-to-use, fully functional lateral flow detection system for the detection of specific protein, DNA or RNA targets. This construct contains all required reagents in an anhydrous format. In some embodiments, the lateral flow device assembly can be completely sealed in order to prevent nucleic acid contamination during use. In this embodiment, the integrity of the device is not compromised.

The disclosed assays and devices of are applicable for the detection of any target molecule, such as a target nucleic acid. The term “target nucleic acid” refers to a nucleic acid targets to be detected by the devices and methods disclosed herein. Sources of target molecules will typically be isolated from organisms and pathogens such as viruses and bacteria. Typical analytes may include nucleic acid fragments including DNA, RNA or synthetic analogs thereof. Additionally, it is contemplated that targets may also be from synthetic sources.

The disclosed assays and devices can detect a target molecule obtained from a variety of samples. Thus, the term “sample” or “test sample” as used herein refers to any fluid sample potentially containing a target nucleic acid. Samples may include biological samples derived from agriculture sources, bacterial and viral sources, and from human or other animal sources, as well as other samples such as waste or drinking water, agricultural products, processed foodstuff, air, etc. Examples of biological samples include blood, stool, sputum, mucus, serum, urine, saliva, teardrop, tissues such as biopsy samples, histological tissue samples, and tissue culture products, agricultural products, waste or drinking water, foodstuff, air, etc. The disclosed assays are useful for the detection of molecules corresponding to certain diseases or conditions such as genetic defects, as well as monitoring efficacy in the treatment of contagious diseases, but is not intended to be limited to these uses.

### Oligonucleotides

Disclosed herein are oligonucleotides for use as marker oligonucleotides, immobilization oligonucleotides, and control probes. In each of these embodiments, the oligonucleotides can be modified to increase stability of nucleic acid half-life and nuclease resistance, such as one or more modifications or substitutions to the nucleobases, sugars, or linkages of the polynucleotide. For example, the polynucleotide can be custom synthesized to contain properties that are tailored to fit a desired use. Common modifications include, but are not limited to use of locked nucleic acids, unlocked nucleic acids (UNA's), morpholinos, peptide nucleic acids (PNA), phosphorothioate linkages, phosphonoacetate, linkages, propyne analogs, 2′-O-methyl RNA, 5-Me-dC, 2′-5′ linked phosphodiester linage, Chimeric Linkages (Mixed phosphorothioate and phosphodiester linkages and modifications), conjugation with lipid and peptides, and combinations thereof.

In some embodiment, the polynucleotide includes internucleotide linkage modifications such as phosphate analogs having achiral and uncharged intersubunit linkages (e.g., Sterchak, E. P. et al., Organic Chem., 52:4202, (1987)), or uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8(1):1-7 (2001)). Commercial nucleic acid synthesizers and standard phosphoramidite chemistry are used to make LNAs. Other backbone and linkage modifications include, but are not limited to, phosphorothioates, peptide nucleic acids, tricyclo-DNA, decoy oligonucleotide, ribozymes, spiegelmers (containing L nucleic acids, an apatamer with high binding affinity), or CpG oligomers.

Phosphorothioates (or S-oligos) are a variant of normal DNA in which one of the nonbridging oxygens is replaced by a sulfur. The sulfurization of the internucleotide bond dramatically reduces the action of endo- and exonucleases including 5′ to 3′ and 3′ to 5′ DNA POL 1 exonuclease, nucleases S1 and P1, RNases, serum nucleases and snake venom phosphodiesterase. In addition, the potential for crossing the lipid bilayer increases. Because of these important improvements, phosphorothioates have found increasing application in cell regulation. Phosphorothioates are made by two principal routes: by the action of a solution of elemental sulfur in carbon disulfide on a hydrogen phosphonate, or by the more recent method of sulfurizing phosphite triesters with either tetraethylthiuram disulfide (TETD) or 3H-1,2-bensodithiol-3-one 1,1-dioxide (BDTD). The latter methods avoid the problem of elemental sulfur's insolubility in most organic solvents and the toxicity of carbon disulfide. The TETD and BDTD methods also yield higher purity phosphorothioates. (See generally Uhlmann and Peymann, 1990, Chemical Reviews 90, at pages 545-561 and references cited therein, Padmapriya and Agrawal, 1993, Bioorg. & Med. Chem. Lett. 3, 761).

Peptide nucleic acids (PNA) are molecules in which the phosphate backbone of oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that is similar to oligonucleotides, but are achiral and neutrally charged molecules. Peptide nucleic acids are typically comprised of peptide nucleic acid monomers. The heterocyclic bases can be any of the standard bases (uracil, thymine, cytosine, adenine and guanine) or any of the modified heterocyclic bases described below. A PNA can also have one or more peptide or amino acid variations and modifications. Thus, the backbone constituents of PNAs may be peptide linkages, or alternatively, they may be non-peptide linkages. Examples include acetyl caps, amino spacers such as 8-amino-3,6-dioxaoctanoic acid (referred to herein as 0-linkers), and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

In some embodiments, the polynucleotide includes one or more chemically-modified heterocyclic bases including, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives, 4-acetylcytosine, 8-hydroxy-N-6-methyladenosine, aziridinylcytosine, 5-(carboxyhydroxylmethyl) uracil, 5-bromouracil, 5-carboxymethylaminomethyl-2-thiouracil, 5-carboxymethylaminomethyluracil, dihydrouracil, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to O-methyl, amino-, and fluoro-modified analogs. Inhibitory RNAs modified with 2′-flouro (2′-F) pyrimidines appear to have favorable properties in vitro (Chiu and Rana 2003; Harborth et al. 2003). Moreover, one report recently suggested 2′-F modified siRNAs have enhanced activity in cell culture as compared to 2′-OH containing siRNAs (Chiu and Rana 2003). 2′-F modified siRNAs are functional in mice but that they do not necessarily have enhanced intracellular activity over 2′-OH siRNAs.

In some embodiments the polynucleotide include one or more sugar moiety modifications, including, but are not limited to, 2′-O-aminoethoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA).

### Nucleic Acid Aptamers

In some embodiments, the disclosed marker and/or immobilization oligonucleotides are nucleic acid aptamers, e.g. that specifically bind a protein target molecule. Nucleic acid aptamers are typically oligonucleotides ranging from 15-50 bases in length that fold into defined secondary and tertiary structures, such as stem-loops or G-quartets. Nucleic acid aptamers preferably bind the target molecule with a K_{d} less than 10⁻⁶, 10⁻⁸, 10⁻¹⁰, or 10⁻¹². Nucleic acid aptamers can also bind the target molecule with a very high degree of specificity. It is preferred that the nucleic acid aptamers have a K_{d} with the target molecule at least 10, 100, 1000, 10,000, or 100,000 fold lower than the K_{d} of other non-targeted molecules.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. An aptamer database containing comprehensive sequence information on aptamers and unnatural ribozymes that have been generated by in vitro selection methods is available at aptamer.icmb.utexas.edu.

A nucleic acid aptamer generally has higher specificity and affinity to a target molecule than an antibody. Accordingly, a nucleic acid aptamer can specifically, directly, and firmly bind to a target molecule. Since the number of target amino acid residues necessary for binding may be smaller than that of an antibody, for example, a nucleic acid aptamer is superior to an antibody, when selective suppression of functions of a given protein among highly homologous proteins is intended.

Non-modified nucleic acid aptamers are cleared rapidly from the bloodstream, with a half-life of minutes to hours, mainly due to nuclease degradation and clearance from the body by the kidneys, a result of the aptamer's inherently low molecular weight. This rapid clearance can be an advantage in applications such as in vivo diagnostic imaging. However, several modifications, such as 2′-fluorine-substituted pyrimidines, polyethylene glycol (PEG) linkage, etc. are available to increase the serum half-life of aptamers to the day or even week time scale.

Another approach to increase the nuclease resistance of aptamers is to use a Spiegelmer. Spiegelmers are ribonucleic acid (RNA)-like molecules built from the unnatural L-ribonucleotides. Spiegelmers are therefore the stereochemical mirror images (enantiomers) of natural oligonucleotides. Like other aptamers, Spiegelmers are able to bind target molecules such as proteins. The affinity of Spiegelmers to their target molecules often lies in the pico-to nanomolar range and is thus comparable to antibodies. In contrast to other aptamers, Spiegelmers have high stability in blood serum since they are less susceptible to be cleaved hydrolytically by enzymes. Nonetheless, they are excreted by the kidneys in a short time due to their low molar mass. Unlike other aptamers, Spiegelmers may not be directly produced by the SELEX method. This is because L-nucleic acids are not amenable to enzymatic methods, such as polymerase chain reaction. Instead, the sequence of a natural aptamer identified by the SELEX method is determined and then used in the artificial synthesis of the mirror image of the natural aptamer.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Detection of miRNA21 by Fluorescence Immunochromatography

Two LNA based probe sandwich method for cancer diagnosis

Step1: Test hybridization study of fluorescent-LNA and miRNA21

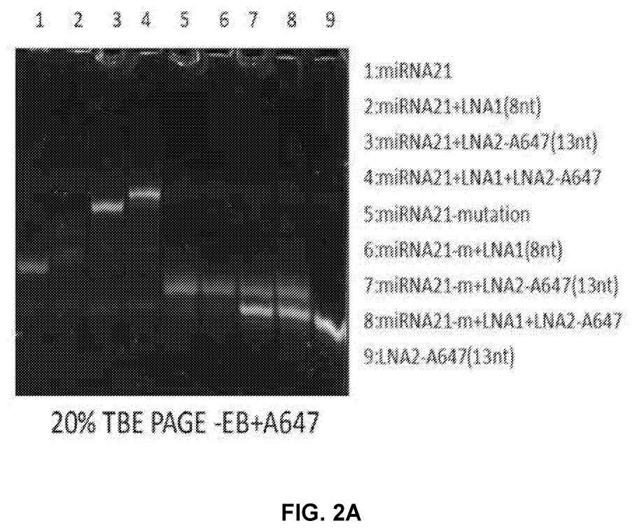

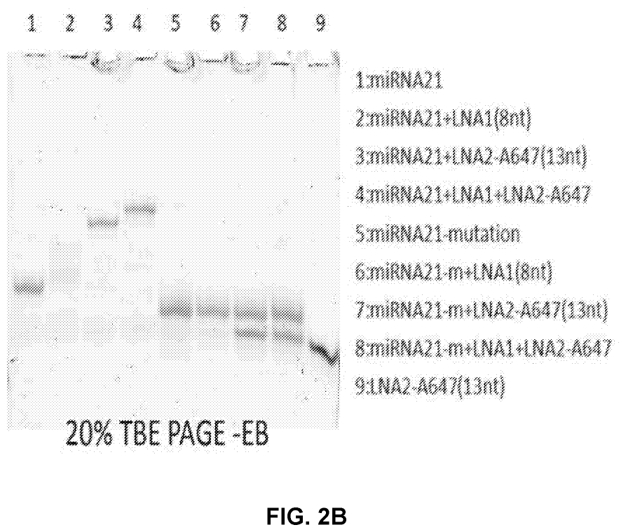

Result: RT for 15 min before loading to 20% TBE Gel. From the gel, we can see LNA-miR21 band after adding two LNA probes. This hybridization is specific since miRNA21 mutation strands did not bind to LNA (FIGS. 2A and 2B).

Step2: Test the flow of fluorescent-RNA on the nitrocellulose membrane

The NC I used is: Whatman Fast Flow, High Performance Plus (FF120 HP Plus). It's from GE company.

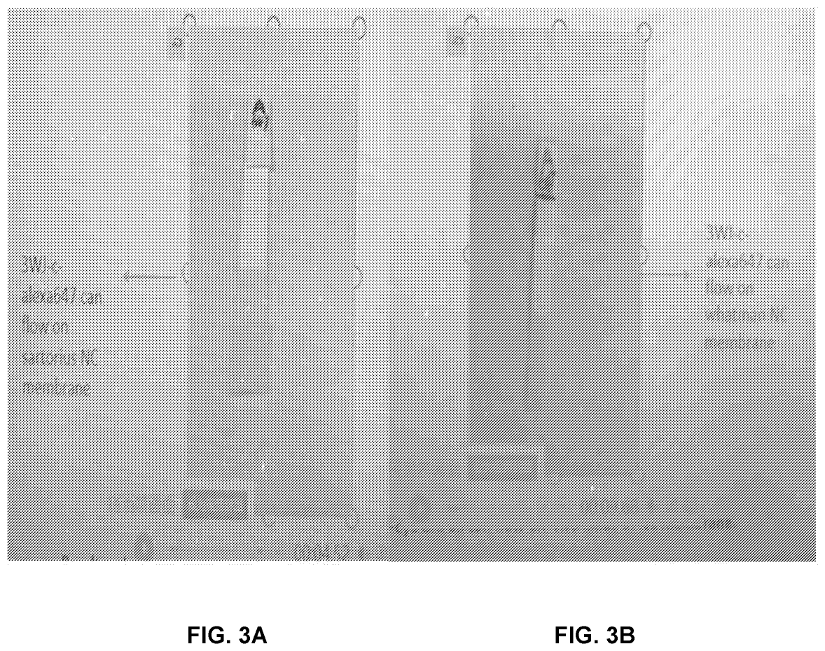

Result: 3WJ-c-alexa647 and 3WJ-b-cy5 are all can flow on two kinds of NC membrane (FIGS. 3A and 3B).

Step3: Test RNA anchored on nitrocellulose membrane

(1) confirm that Cholesterol helps RNA anchor to nitrocellulose membrane.

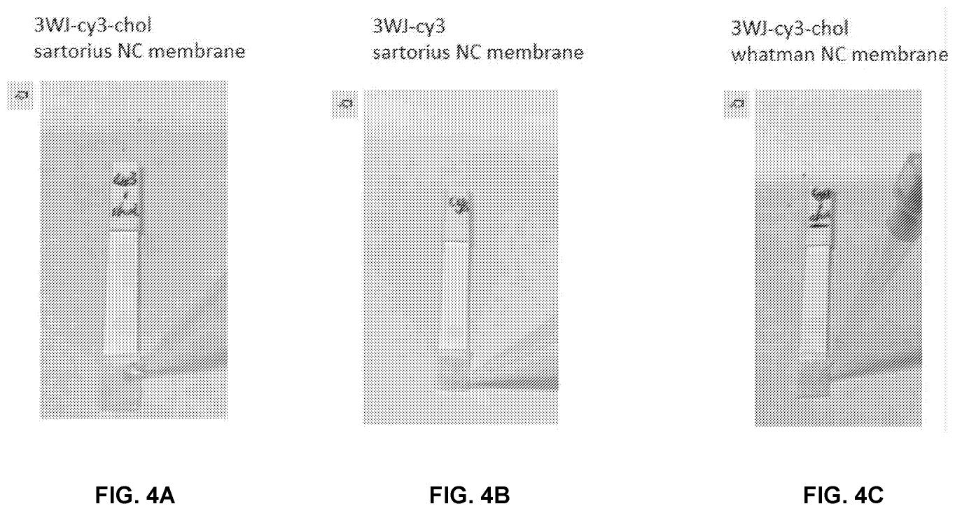

Result: 3WJ-cy3-chol can anchor on whatman NC membrane. whatman NC membrane adsorption is stronger than Sartorius NC membrane (FIGS. 4A to 4C).

Detection of the anchor efficiency of cholesterol at 3WJ different locations

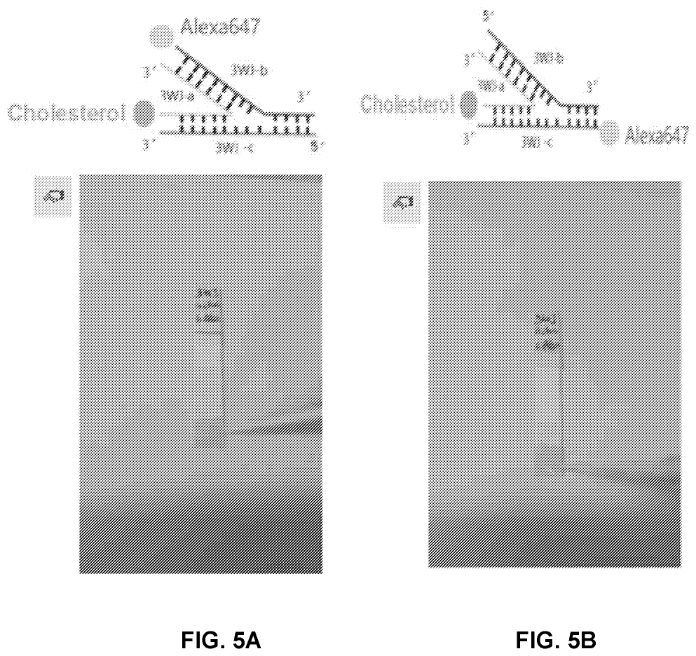

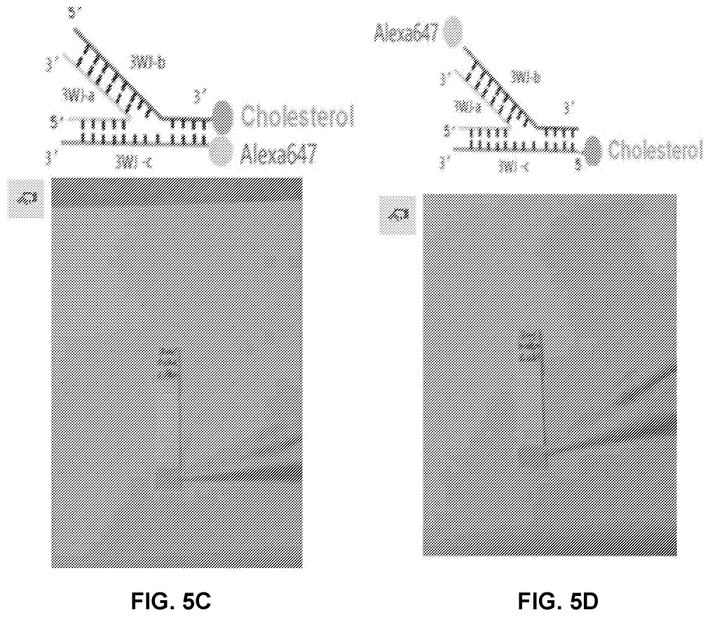

Result: The results showed that cholesterol all has a anchor efficiency at different positions of 3WJ (FIGS. 5A to 5D).

Detecting the effect of different amounts of cholesterol on anchoring

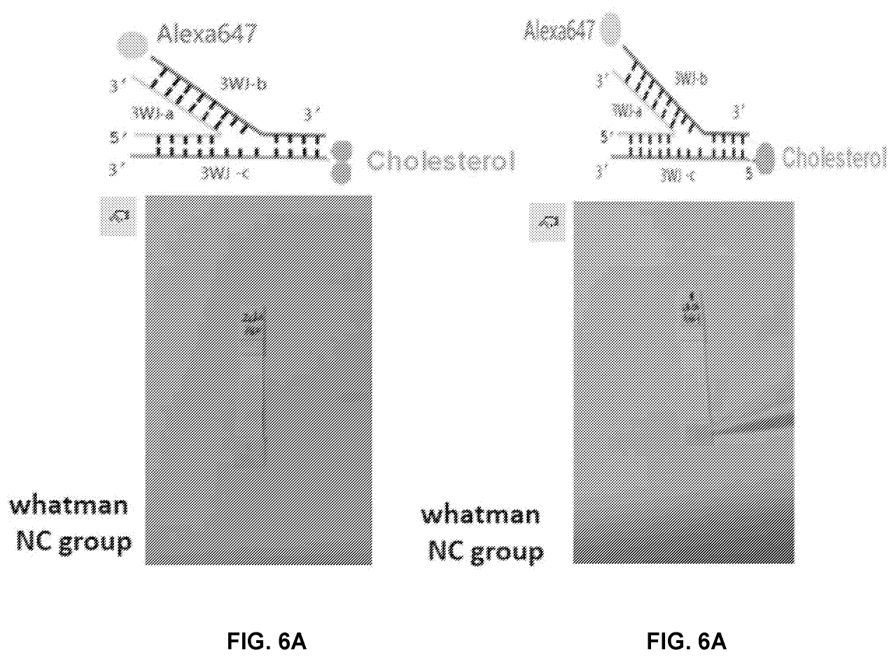

Result: In order to study whether the amount of cholesterol affects its fixation effect, we compared the anchor efficiency of 3WJ-2chol and 3WJ-1chol, and the results showed that there was no significant difference in the immobilization efficiency in whatman NC membrane and all can have the anchor efficiency (FIGS. 6A and 6B).

Step4: Test miR21+LNA2-AF647 bind with LNA1 and LNA3 on NC membrane

(1) Test miR21+LNA2-AF647 bind with LNA1 and LNA3 on NC membrane (Work with Hongran)

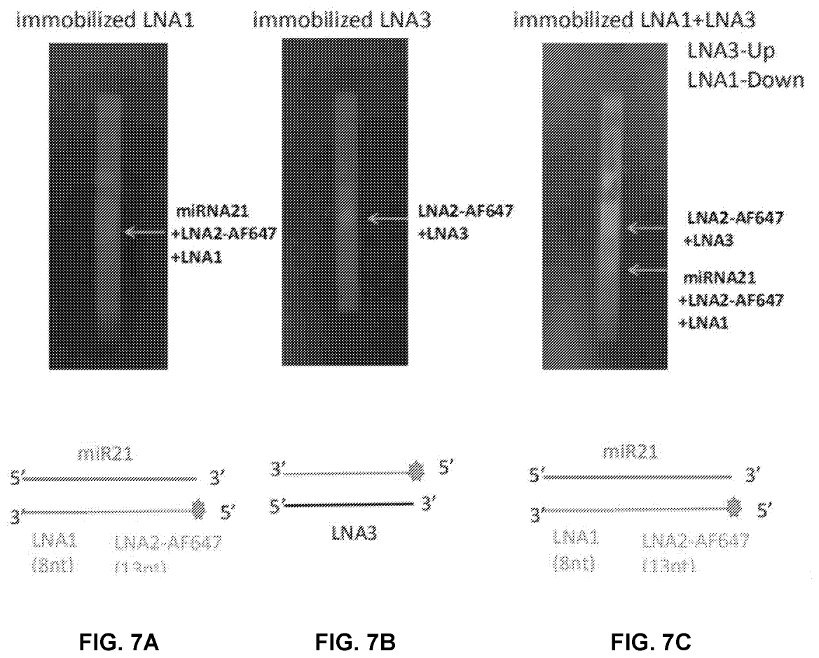

Result: Due to lack of negative controls, specific binding cannot be determined (FIG. 7A to 7C).

(2) Test LNA2-AF647 non-specific bind with LNA1 on NC membrane (Hongran's Work)

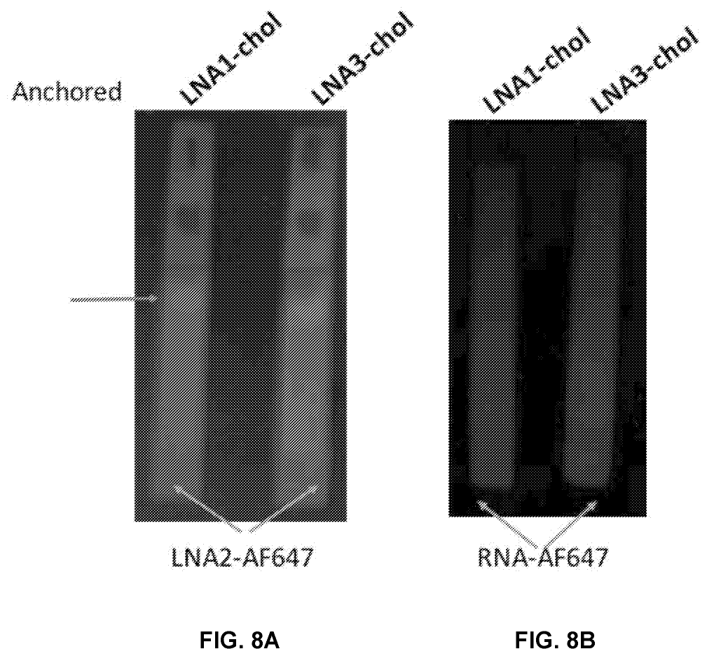

Result: We found false positive signal in lateral flow test that LNA1 and LNA2 have reaction and show signal (Left figure). To figure out this problem, we firstly want to see whether it is due to AF647 fluorophore non-specific reaction with cholesterol. So another RNA strand with AF647 was used for test. It did not show that false positive signal (Right figure). Therefore, AF647 fluorophore is not the reason for non-specific signal (FIGS. 8A and 8B).

(3) Test whether cholesterol affects LNA2-AF647 non-specific bind with LNA1 on NC membrane (Hongran's Work)

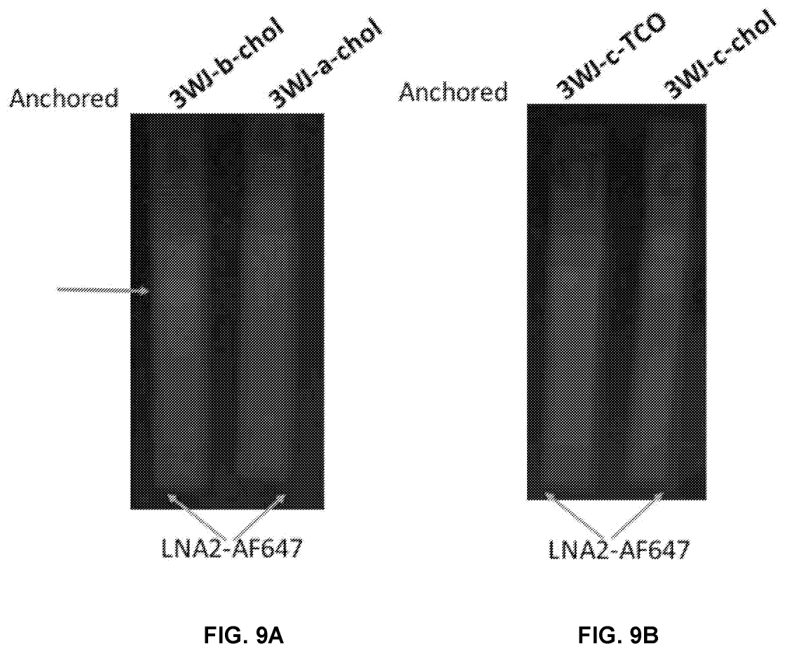

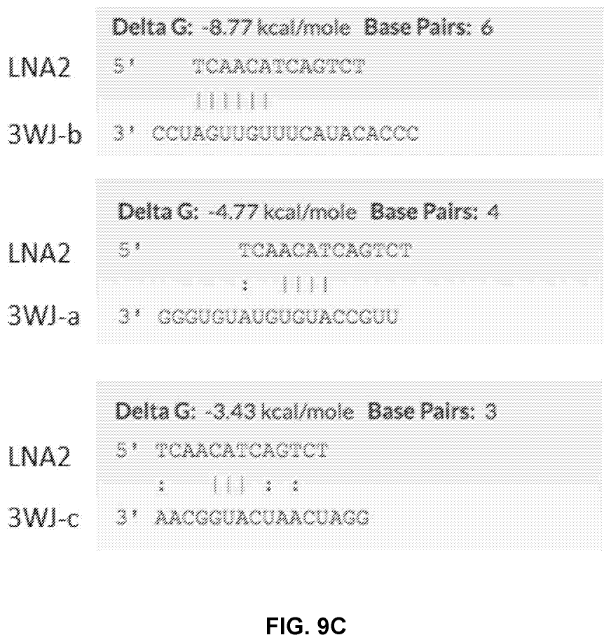

Result: we suspect cholesterol may be the reason for the false positive reason. So other cholesterol-RNA strands were used to anchor on membrane. The result showed no false positive signal was found on NC except 3WJ-b-chol. After checking the sequence, we found there're 6 bp between LNA2-AF647 and 3WJ-b. If we look at LNA1 and LNA2 sequence, we found they have 3 bp perfect match (last slide). LNA modification increase thermodynamic stability a lot and may cause non-specific binding. So the signal is very likely from non-specific base pairings rather than cholesterol issue (FIGS. 9A to 9C).

(4) Change LNA2 to 2′F RNA to resolve non-specific binding (Hongran's Work)

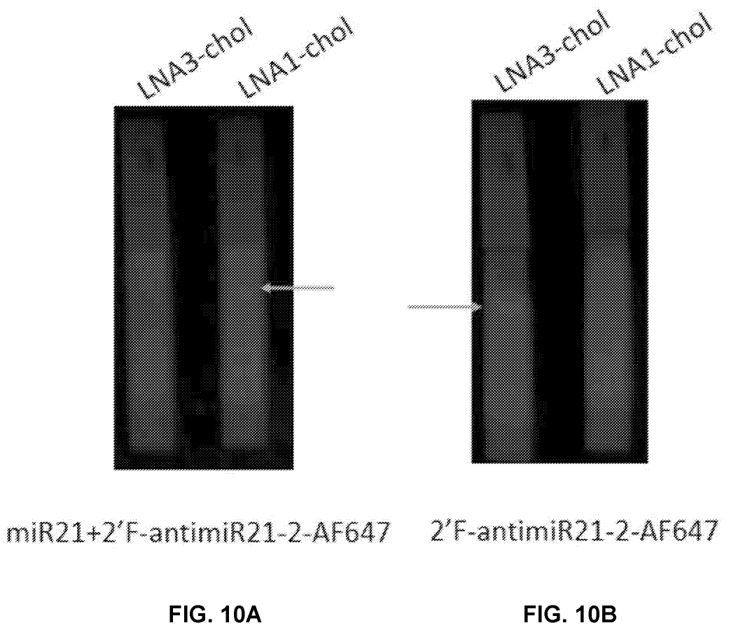

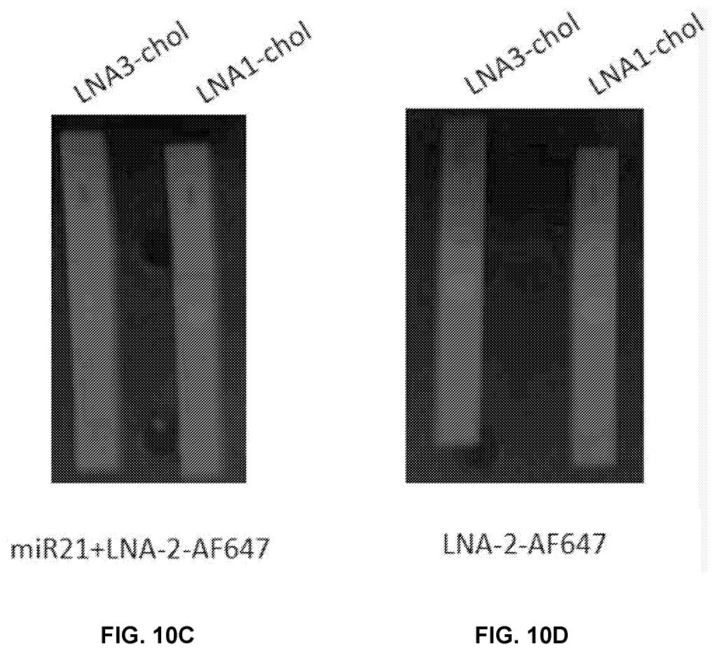

Result: The result showed signal was detected within pre-mix miR21+2′F-antimiR21-2-AF647 group. In comparison, 2′F modified anti21 strand without miR21 did not show false positive signal on LNA1 anchored NC membrane. LNA2-AF647 without miR21 still showed strong non-specific signal on LNA1-chol NC membrane. 2′F modified strand can eliminate the non-specific reaction with LNA1 (FIGS. 10A to 10D).

miR21-S+2′F antimiR21-AF647 miR21-S+2′F antimiR21-AF647

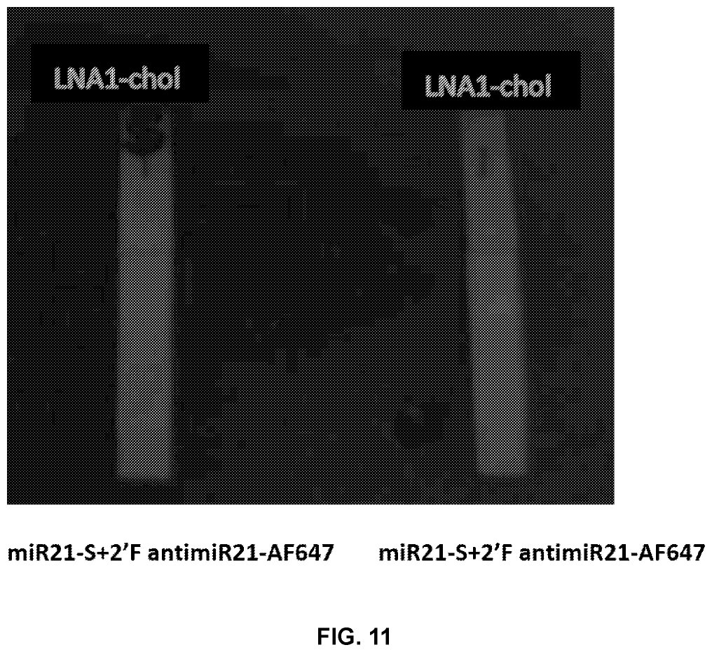

Result: 2′F anti21-AF647 specific bind with miR21 and no bind with miR21-S (FIG. 11).

Next Plan

Detection the Sensitivity of miRNA21 by fluorescence immunochromatography

### Example 2: Detection of miRNA21 by Colloidal Gold Immunochromatography

Two LNA based probe sandwich method for cancer diagnosis

Step1: Electrophoresis detection of colloidal gold label LNA2

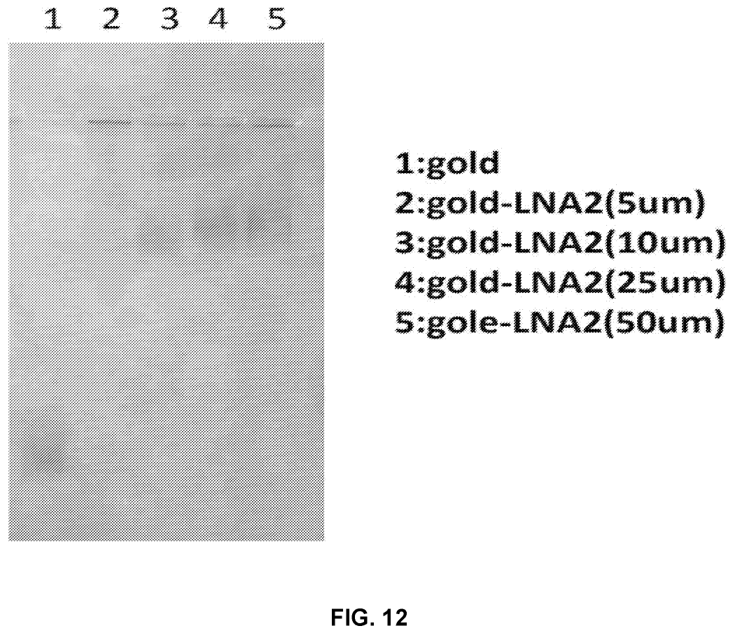

Result: different concentration LNA2 (5 um, 10 um, 25 um, 50 um) separately add 2 μl of 1 mM TCEP 1 h, then added 1 ml gold, string 1 h, add 10 ul 10% BSA for 45 min, add 20 ul NaCl for 1 h, then 4° C. overnight, then 2% agarose gel electrophoresis, 100 v 1 h. The results show that the electrophoresis speed of gold is slow after labeling LNA2, the higher the concentration, the slower the electrophoresis speed (FIG. 12).

Step2: Detecting the binding ability of labelling different concentration of colloidal gold-LNA2 to LNA3 on NC membrane

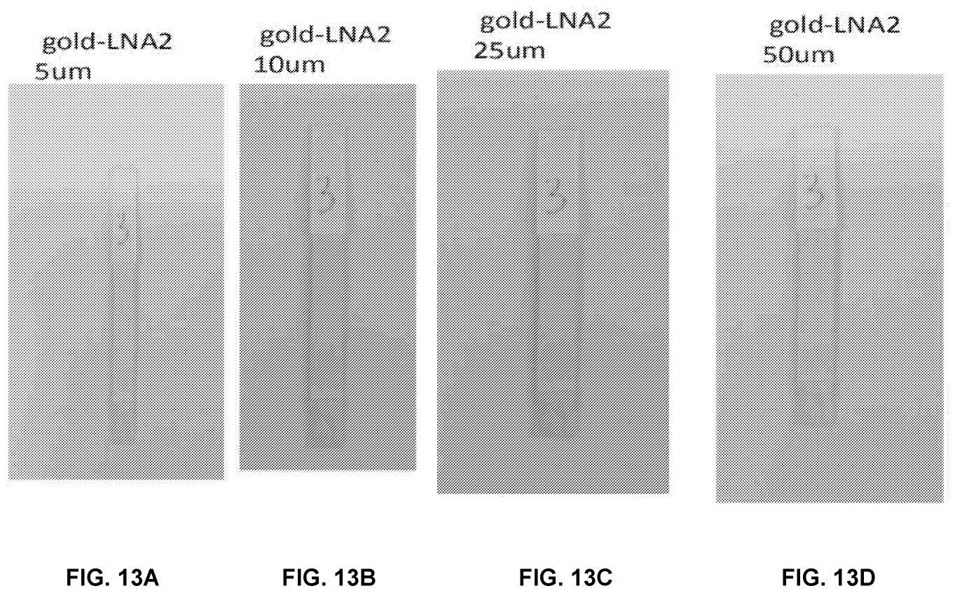

Result: LNA3-Chol (40 uM) coated on NC membrane and add seperately gold-LNA2 (5 um, 10 um, 25 um, 50 um), The signal does not increase with increasing gold-LNA2 concentration (FIGS. 13A to 13D).

Step3: Detecting the binding ability of colloidal gold-LNA2 to anchored different concentration of LNA3 on NC membrane

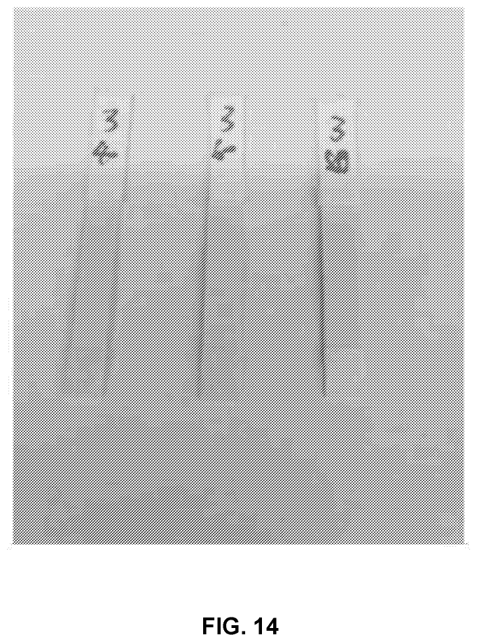

Result: LNA3-Chol (40 uM, 60 uM, 80 uM) coated on NC membrane and add gold-LNA2 (50 um), The signal does not increase with increasing anchored-LNA3 concentration (FIG. 14).

Problem analysis: It is speculated that the gold labeling step is not efficient, and further conditions need to be explored, such as the concentration of TCEP, the time of labeling, and the concentration of sodium chloride. Finding the best labeling conditions.

### Example 3: Two LNA for miRNA Diagnosis

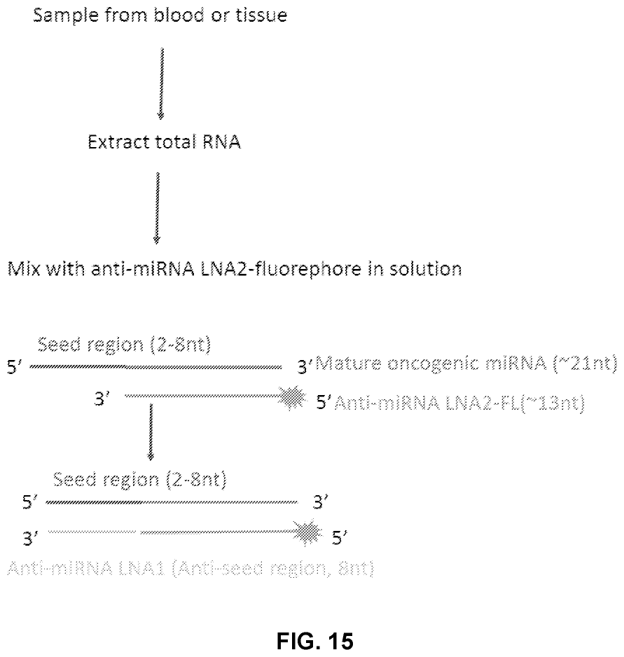

FIG. 15 shows a two LNA based probe sandwich method for cancer diagnosis. The sequences used are miRNA17: 5′-CAAAGUGCUUACAGUGCAGGUA-3′ (SEQ ID NO:1), miRNA21: 5′-UAGCUUAUCAGACUGAUGUUGA-3′ (SEQ ID NO:2), miRNA155: 5′-UUAAUGCUAAUCGUGAUAGGGGU-3′ (SEQ ID NO:3), anti-miR21 LNA2-Alexa647: 5′-Cy5-+T+C+A+A+C+A+T+C+A+G+T+C+T-3′ (SEQ ID NO:4), and anti-miR21 LNA1: 5′-+G+A+T+A+A+G+C+T-3′.

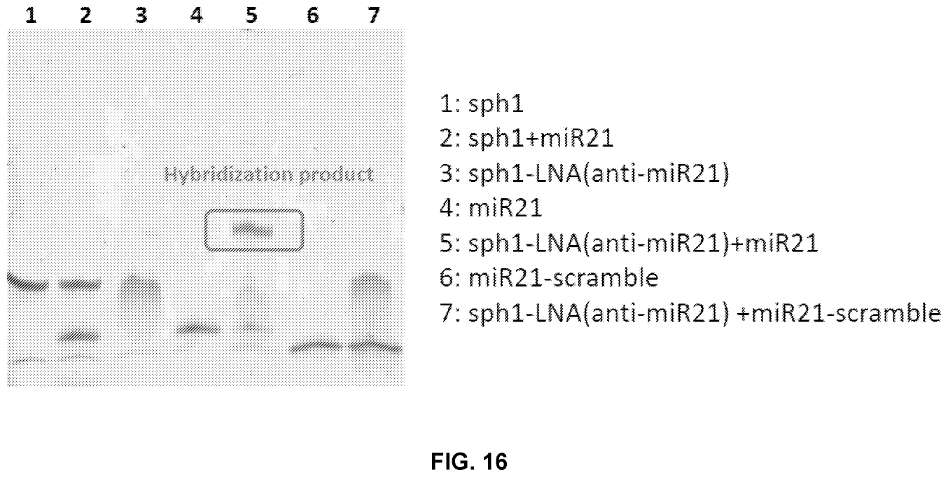

FIG. 16 shows a hybridization study of LNA-RNA. This shows that miR21 can hybridize with 8nt LNA modified anti-miR21 upon mix at room temperature, while no hybridization was seen for miR-scramble control.

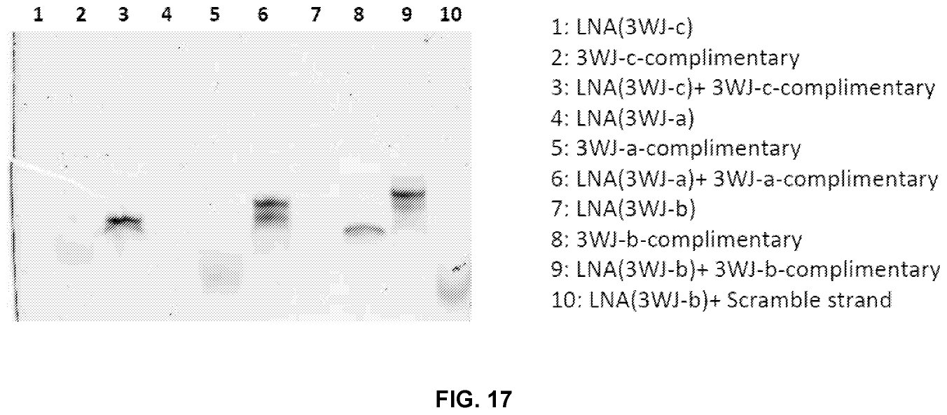

FIG. 17 shows a hybridization study of LNA-RNA. This shows 2′F modified 3WJ-a, 3WJ-b, 3WJ-c-complimentary strands can hybridize with LNA-a, LNA-b, LNA-c at room temperature respectively.

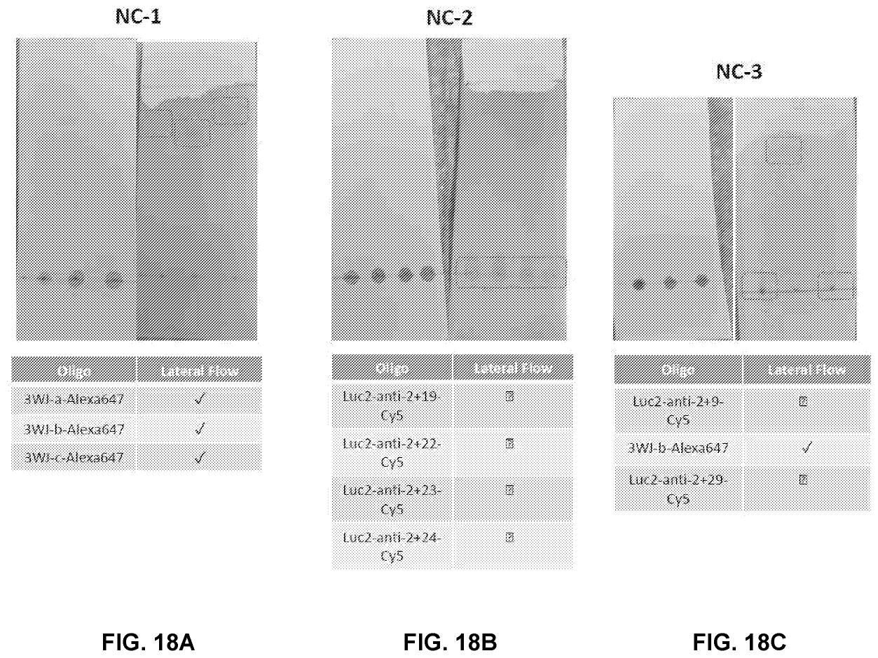

FIGS. 18A to 18C show lateral flow of RNA samples on new NC membranes NC-1 (FIG. 18A), NC-2 (FIG. 18B), and NC-3 (FIG. 18C). Whatman Fast Flow, High Performance Plus (FF120 HP Plus) Nitrocellulose membrane Develop solution: dd-H₂O.

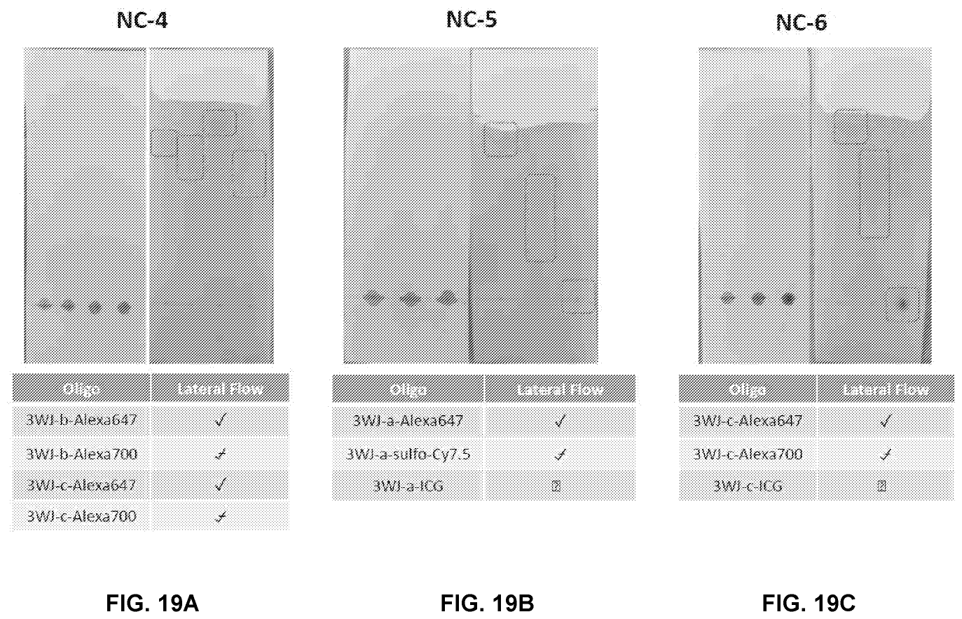

FIGS. 19A to 19C show lateral flow of RNA samples on new NC membranes NC-4 (FIG. 19A), NC-5 (FIG. 19B), and NC-6 (FIG. 19C). Different fluorophore was attached on RNA strands for lateral flow test. The data shows some hydrophobic dye (ICG, Cy5) conjugated RNA cannot migrate on NC. Less hydrophobic Alexa647 conjugated RNA strand can migrate on NC.

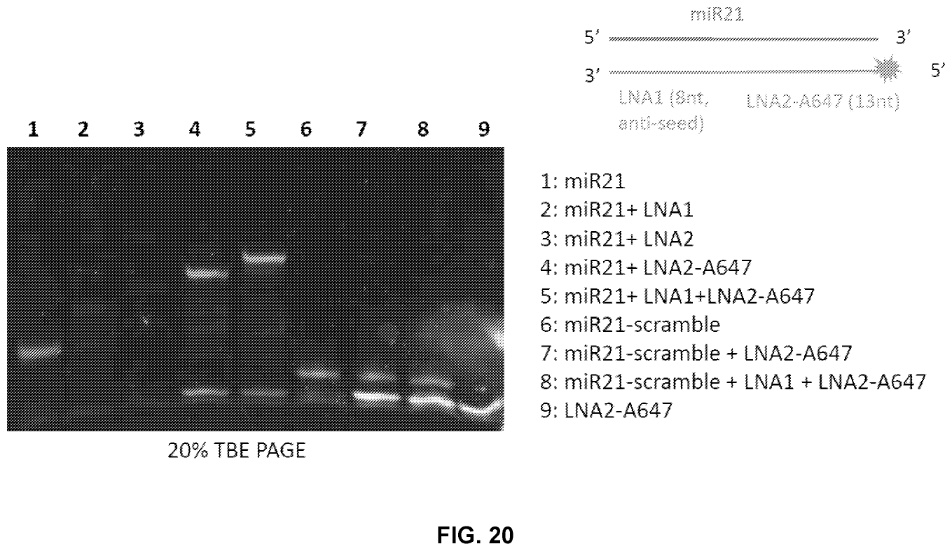

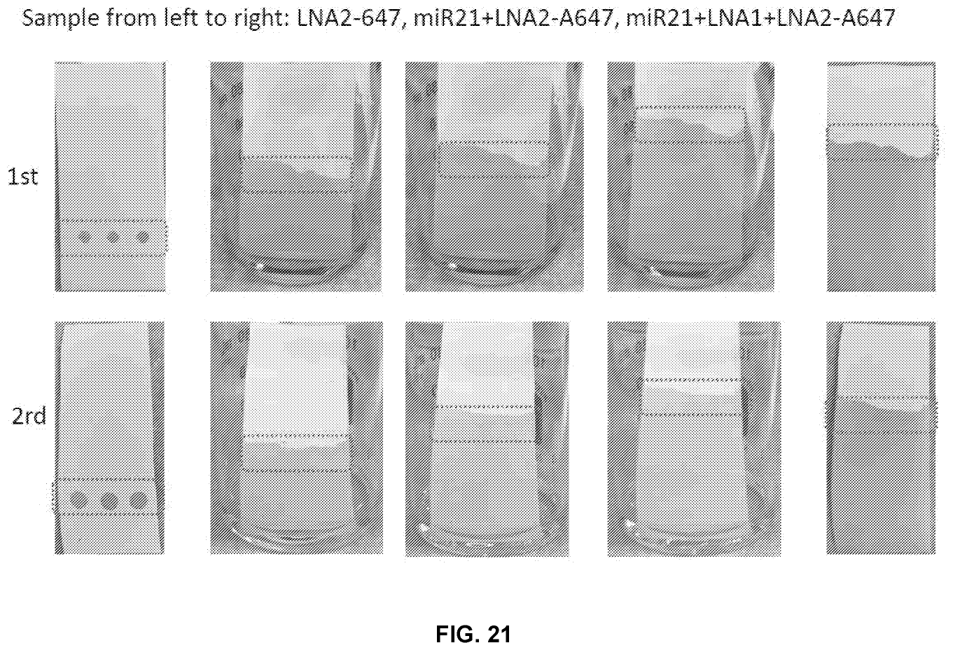

FIG. 20 shows LNA-A647 hybridization. This shows miR21 can hybridize with two LNA modified anti-miR21 probes upon mix at room temperature, while no hybridization was seen for miR-scramble control.

FIG. 21 shows LNA-A647 lateral flow on NC membrane. Both LNA-A647 and miR21-LNA complex can run through the NC membrane.

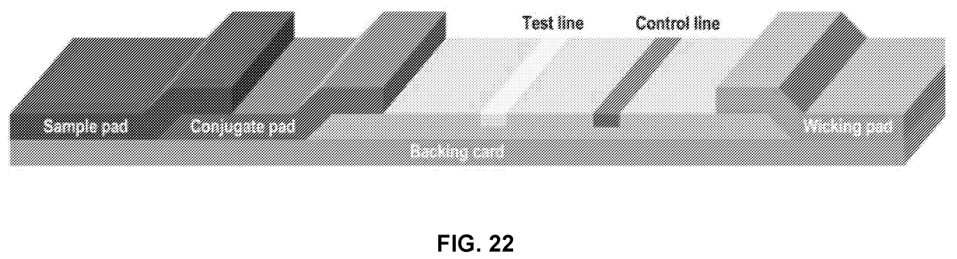

FIG. 22 shows an example lateral flow assay development device, which contains a sample pad, conjugated pad, nitrocellulose membrane, and a wick/absorbent pad. Each component overlaps with the next by 1-2 mm so the sample can move through via capillary force.

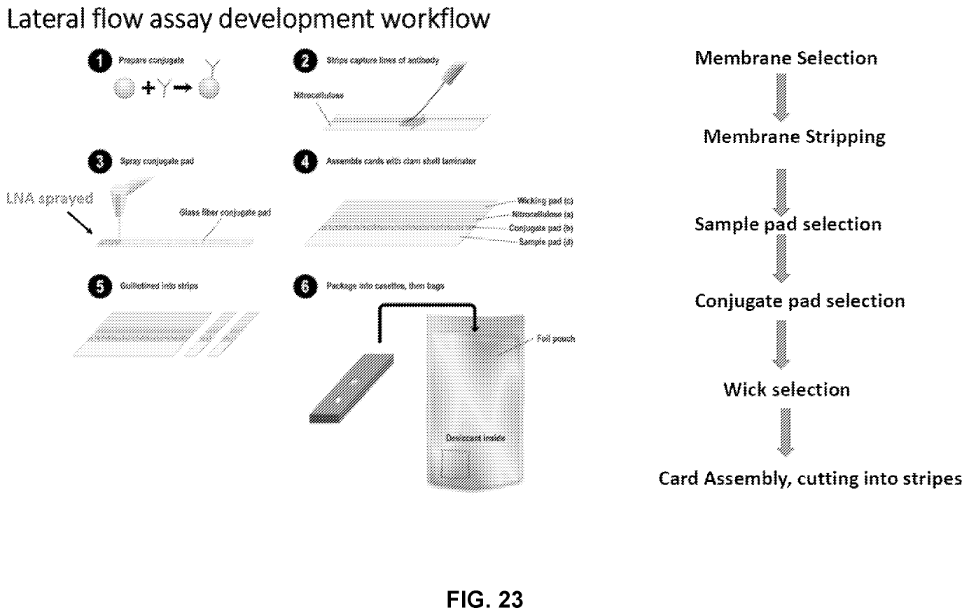

FIG. 23 shows an example of a lateral flow assay development workflow, consisting of conjugate preparation (1), striping of capture lines (2), spraying conjugate pad (3), assembly of cards (4), strip cutting (5), and packaging into cassettes (6).

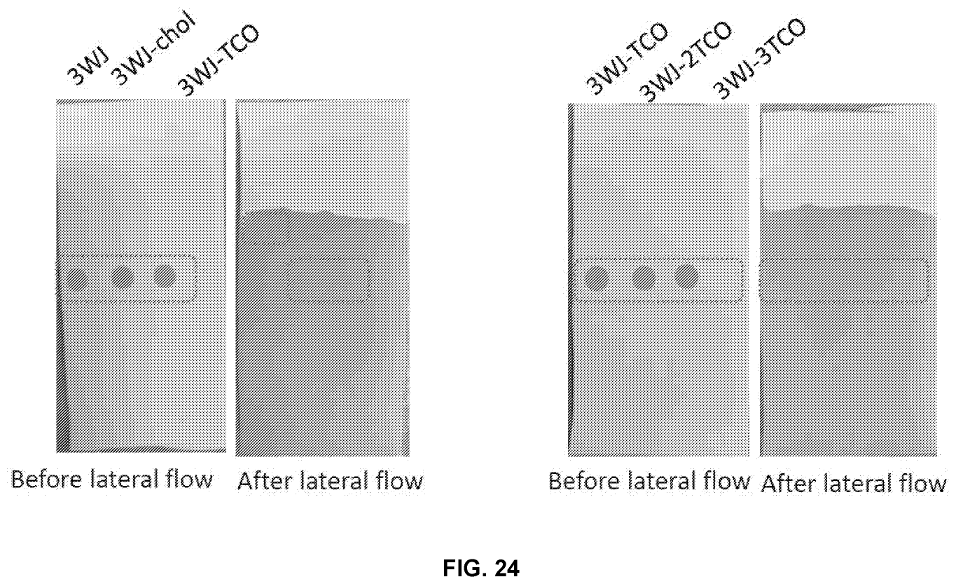

FIG. 24 shows RNA anchor on NC membrane by RNA lipid. This experiment was to test whether RNA-lipid can be immobilized to NC membrane by hydrophobic force. 3WJ-Alexa647 and 3WJ-lipid-Alexa647 were assembled first. Cholesterol was incorporated as well as 1/2/3 tocopherol (TCO) as lipid module. The samples were loaded to NC membrane in the middle and dd-water was used for developing solution. After the lateral flow, all 3WJ-lipid group did not migrate while 3WJ migrate within water. It shows potential to use RNA-lipid as immobilization approach in NC membrane.

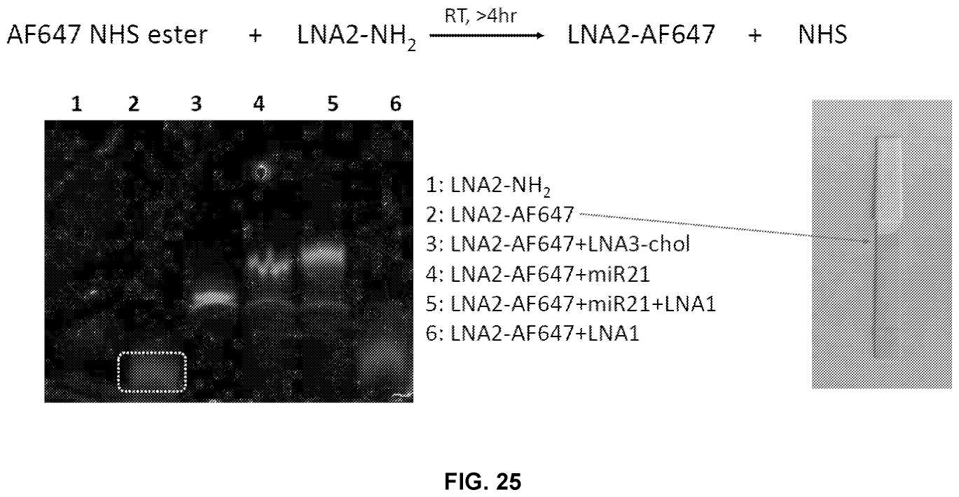

FIG. 25 shows conjugate of AF647 to LNA2 tested for lateral flow on NC membrane. The LNA2-AF647 was synthesized successfully verified by 15% Native gel (Lane 2). Since AF647 has structural difference from Alexa647. Lateral flow was also tested. LNA2-AF647 can also migrate through the NC membrane, which can be used for the next step.

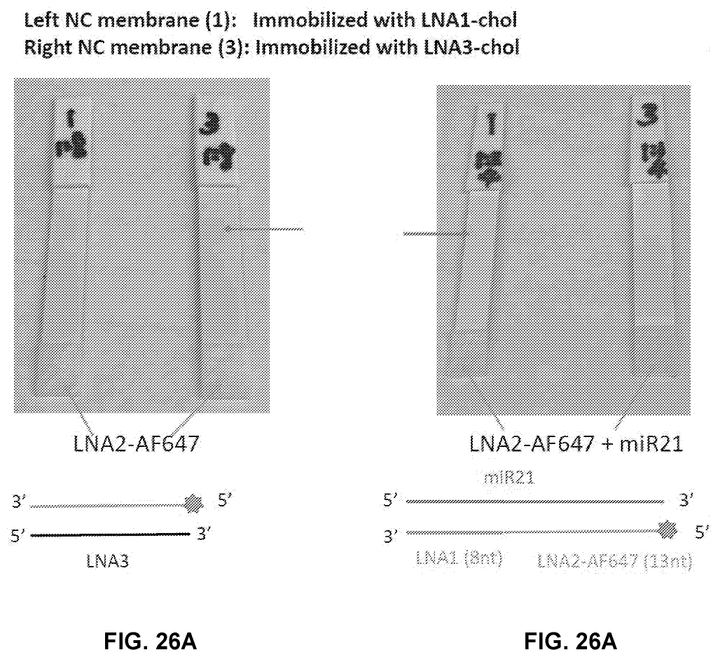

FIGS. 26A and 26B show lateral flow of NC membranes immobilized with LNA1-chol (FIG. 26A) or LNA3-chol (FIG. 26B) for miR21 diagnosis. The test device was composed of a sample pad, NC membrane and wick (from bottom to top). The NC membranes were immobilized by LNA1 (FIG. 26A) and LNA2 (FIG. 26B) in advance. Then, LNA2-AF647 and LNA2-AF647+miR21 were added to sample pad, which can migrate through NC membrane. From the result, it can be seen that LNA2-AF647 reacts with LNA3 but not LNA1 (arrow). LNA2-AF647+miR21 reacts with LNA1 but not with LNA3. However, the color of the spot was very light.

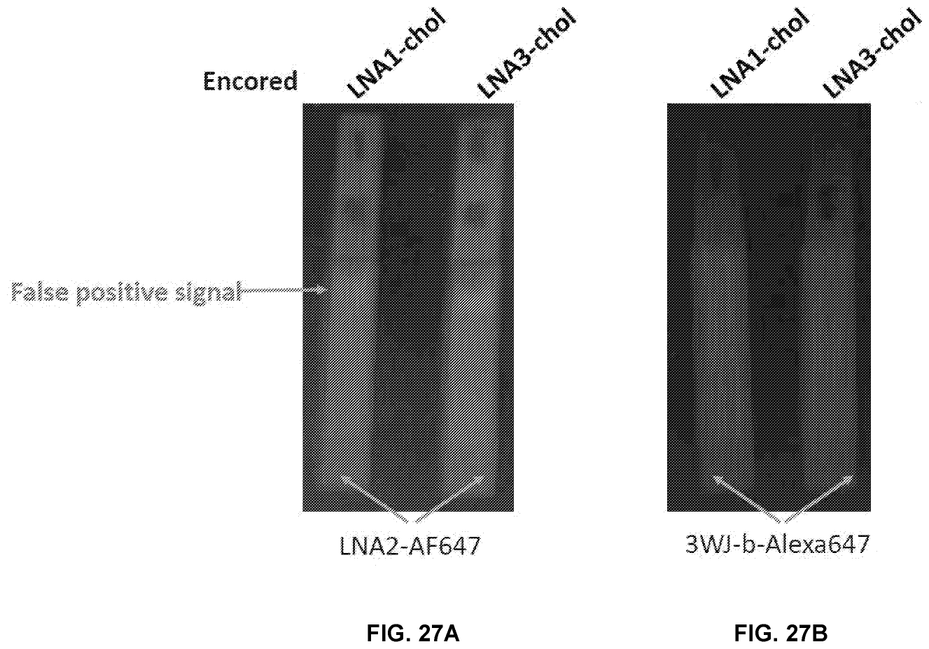

FIGS. 27A and 27B show lateral flow of LNA2-AF647 (FIG. 27A) or 3WJ-b-Alexa647 (FIG. 27B) on LNA1-NC membrane for miR21 diagnosis. From previous results, the positive signal is seen when miR21+LNA2-AF647 was run through LNA1 anchored membrane. However, when lateral flow of LNA2-AF647 was tested on LNA1-NC membrane, a false positive signal appeared. This was repeated several times and the concentration changed, but the signal was still there. When 3WJ-b-Alexa647 was tested as a control, no false signal was shown.

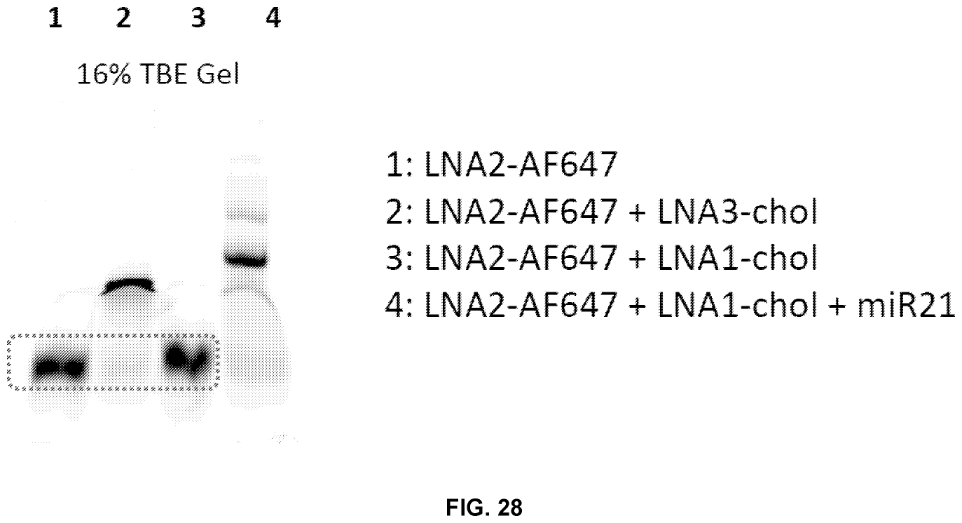

FIG. 28 shows lateral flow of LNA2-AF647 with nothing, LNA3-chol, LNA1-chol, or LNA1-chol+miR21. There was non-specific binding between LNA2-AF647 and LNA1 on NC membrane but not on the gel.

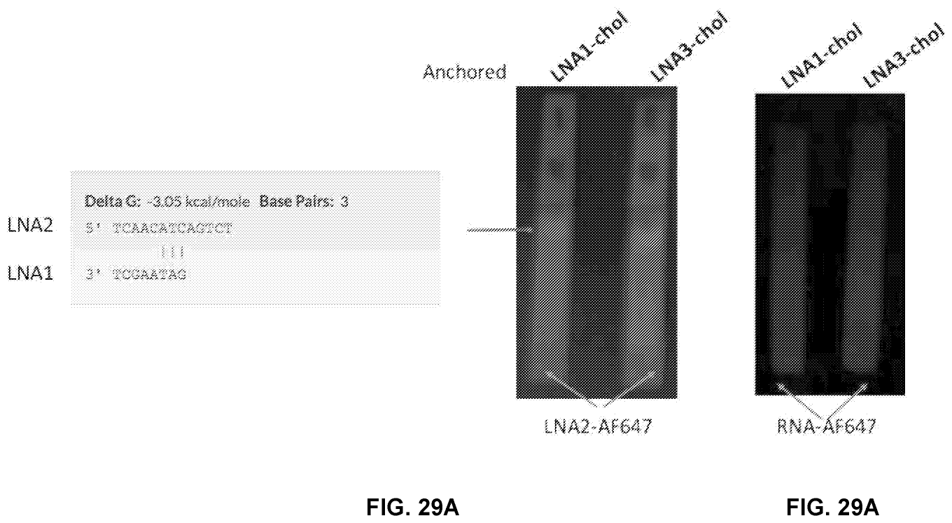

FIG. 29A shows false positive signal in lateral flow test using LNA2-AF647. FIG. 29B shows no signal using another RNA strand with AF647. Therefore, AF647 fluorophore is note the reason for non-specific signal.

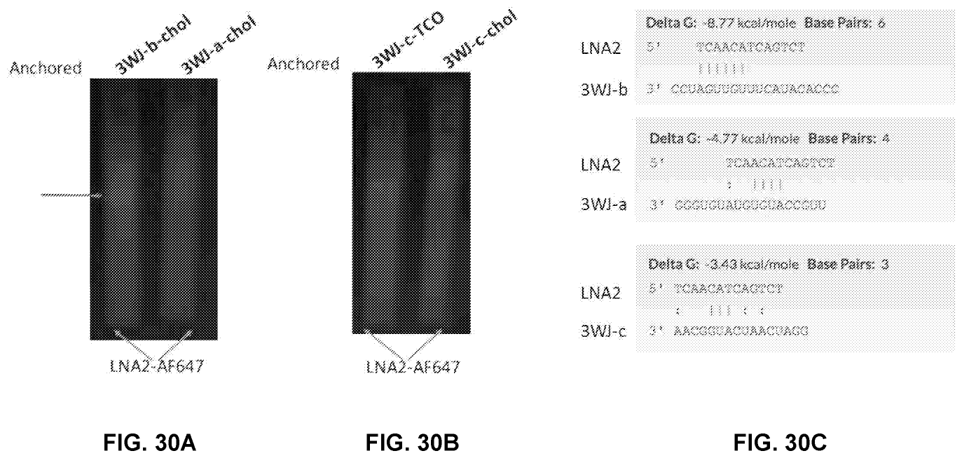

FIGS. 30A to 30C show false positive signal is not from cholesterol. Other cholesterol-RNA strands were used to anchor on membrane. The result showed no false positive signal was found on NC except 3WJ-b-chol. After checking the sequence, it was found that there are 6 bp between LNA2-AF647 and 3WJ-b. The LNA1 and LNA2 sequence have 3 bp perfect match (FIG. 30C). LNA modification increase thermodynamic stability a lot and may cause non-specific binding.

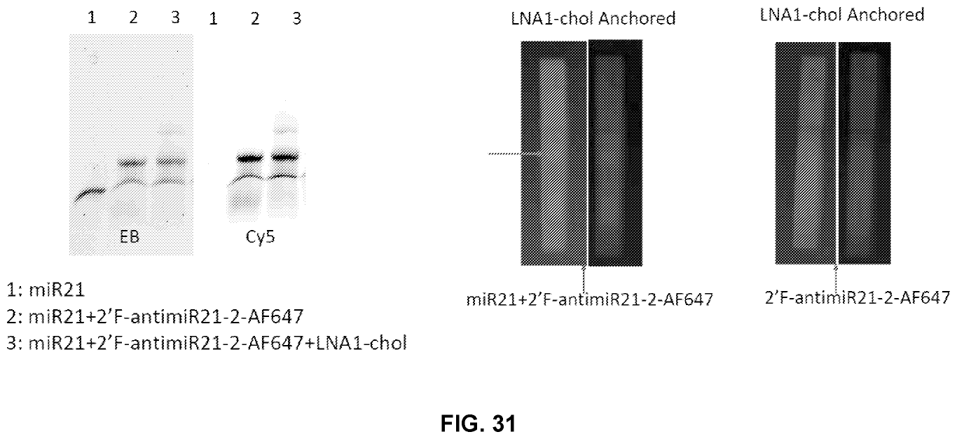

FIGS. 31A to 31C show 2′F modified anti-miR21-2-AF647 can also bind to miR21 efficiently in hybridization study. There was a clear signal within pre-mix miR21-2′F-antimiR21-2-AF647 group. In comparison, 2′F modified anti21 strand without miR21 did not show false positive signal on LNA1 anchored NC membrane.

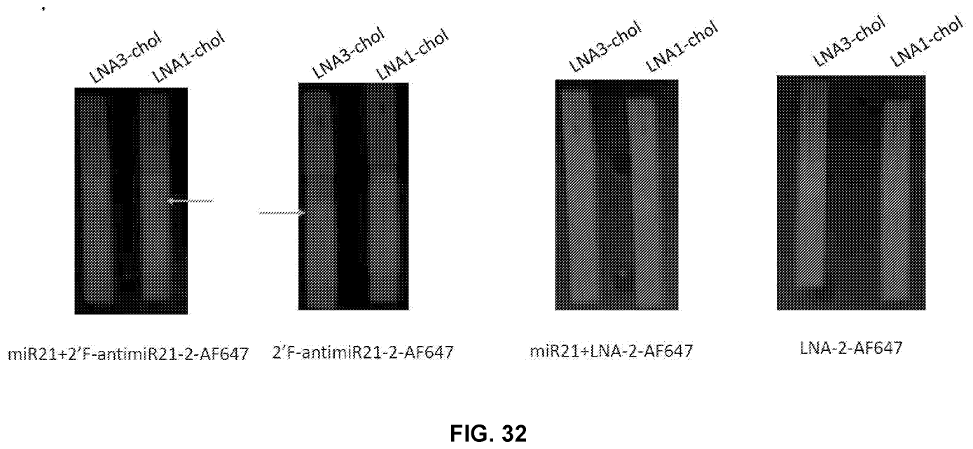

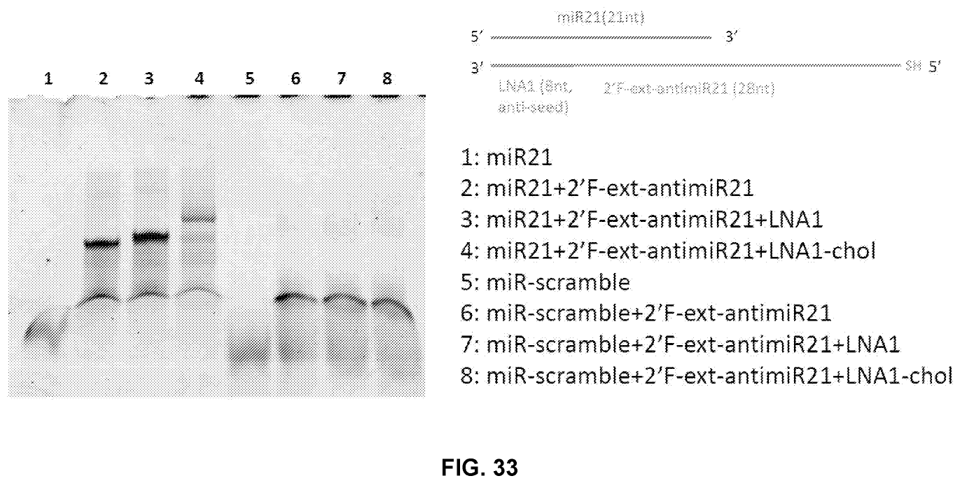

FIGS. 32A to 32D show a repeat of lateral flow test showing 2′F modification can eliminate the non-specific reaction with LNA1. Signal was detected within pre-mix miR21+2′F-antimiR21-2-AF647 group. In comparison, 2′F modified anti21 strand without miR21 did not show false positive signal on LNA1 anchored NC membrane. LNA2-AF647 without miR21 still showed strong non-specific signal on LNA1-chol NC membrane.

FIG. 33 shows hybridization of miR21 with an extended 2′F RNA with miR21. The result showed efficient hybridization of extended 2′F RNA with miR21, as well as with LNA1 at room temperature for 0.5 hr. Scrambled controls did not show the hybridization band. So it is no problem that 2′F modified antimiR21 can be used to bind to miR21. Currently, the main problem is to improve gold conjugation with RNA.

### Example 4: Rapid, Simple, and Low-Cost for Early Diagnosis of COVID-19 by Nuclei Acid Probes on Nitrocellulose Membrane without the Need of Other Equipment

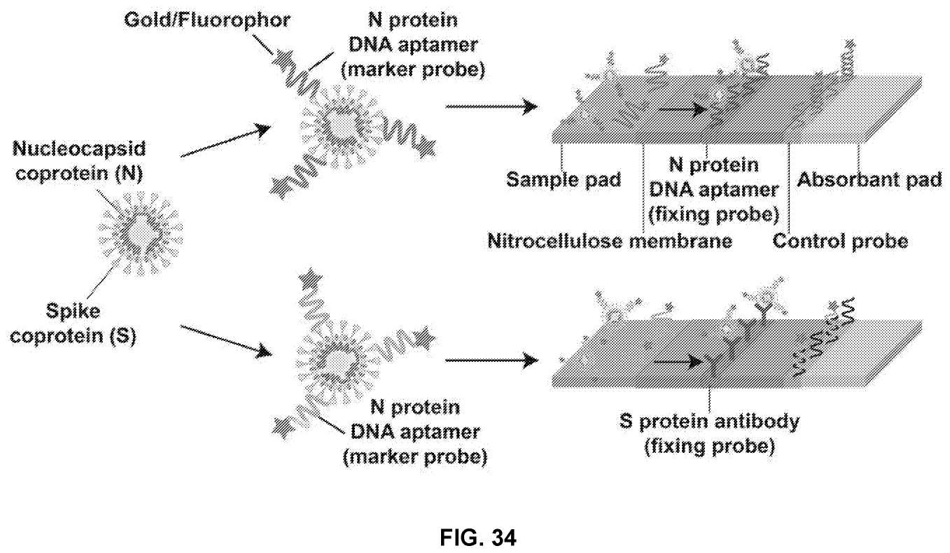

The disclosed method used a first probe that is a gold or fluorophore labeled aptamer (marker probe) to find and label the SARS-CoV-2 virus, and a second probe that is fixed on a nitrocellulose membrane (NC) (fixing probe) to concentrate the labelled virus in a band (FIG. 34). Several microliters of saliva, urine or patient blood taken by needle pinprick when applied to the sample patch containing the marker probe, a DNA aptamer labelled by gold or fluorescent dye. Given the capillary phenomenon, the virus in the blood sample bound with the marker probe will migrate along the filter, ensuring capture by the second fixing probe. This immobilizes it on the filter as a rectangular band. The entire detection process can be completed in several minutes with visible results. No additional equipment is needed. This method can provide a convenient, simple, efficient, and low-cost diagnosis method for COVID-19. The approach is the use of aptamers that bind to one virus or to one protein (to detect the protein as replication by-product).

Design and preparation of DNA aptamer-based marker probe (3 weeks). Four DNA aptamers have been available to bind to four different epitopes of the nucleocapsid (N) proteins of SARS-CoV-2 with high affinity (Table. 1). For this approach, one DNA aptamer is labeled with gold nanoparticles or fluorophore as a marker probe (FIG. 34). The marker probe can be constructed by labeling the aptamer with —SH (or —NH₂) group which is then reacted with gold nanoparticles (or fluorophore-NHS).

TABLE 1
Sequence of four aptamer targets to nucleocapsid protein of SARS-CoV-2.

| Entry | | | | |
|---|---|---|---|---|
| Np- | Sequence (5′-3′) | Ka (1/Ms) | kd (1/s) | Kd (nM) |
| A48 | GCTGGATGTCGCTTACGACAATATTCCTTAGGGGCACCGCTACATTGACACA | 8.80 × 10⁵ | 3.85 × 10⁻⁴ | 0.49 × 0.23 |
| | TCCAGC (SEQ ID NO: 5) | | | |
| A15 | GCTGGATGTTCATGCTGGCAAAATTCCTTAGGGGCACCGTTACTTTGACACA | 2.84 × 10⁵ | 1.16 × 10⁻³ | 4.38 × 1.6 |
| | TCCAGC (SEQ ID NO: 6) | | | |
| A61 | GCTGGATGTTGACCTTTACAGATCGGATTCTGTGGGGCGTTAAACTGACACA | 4.58 × 10⁵ | 1.18 × 10⁻³ | 2.74 × 0.91 |
| | TCCAGC (SEQ ID NO: 7) | | | |
| A58 | GCTGGATGTCACCGGATTGTCGGACATCGGATTGTCTGAGTCATATGACACA | 1.92 × 10⁶ | 1.34 × 10⁻³ | 0.70 × 0.06 |
| | TCCAGC (SEQ ID NO: 8) | | | |

Design and test the fixing and anchoring of the fixing probe on nitrocellulose membrane (3 weeks). To anchor the fixing and control probe on nitrocellulose membrane, lipid molecules such as cholesterol or tocopherol are conjugated to the DNA aptamer. The RNA-lipid complex can be riveted to the nitrocellulose membrane by hydrophobic force for miRNA diagnosis. To test the anchoring ability, a fluorophore labelled complementary oligo can be ran through the nitrocellulose membrane to check whether the fixing probe can “catch” it and show the fluorescence signal. Biotin can also attach to the end of the aptamer, which is capable of binding to streptavidin treated nitrocellulose membrane for anchoring.

The binding affinity of the marker probe is not a concern since each virus contains tens or hundreds of N proteins. If the binding affinity of the fixing probe is too low, commercially available antibodies (Rabbit anti-SARS-CoV-2 S1 RBD, Rabbit anti-SARS-CoV-2-N protein, Rabbit anti-SARSCoV-2-S2 from RayBiotech) can be used to mount on the nitrocellulose membrane as an alternative fixing probe to enhance binding affinity for binding to the virus.

Study sensitivity and specificity with patient derived saliva, urine, milk, or serum (4.5 months). The sensitivity can be adjusted and optimized. Each of the two aptamers are tested as a marker/fixing probe pair for the detection. The pair with strongest S/B (signal/background) is selected. The concentration of probes is titrated and determined to attain a clear and visible signal. The specificity is studied using the biological samples from infected and uninfected people.

Besides using two aptamers binding to two different sites on a nucleocapsid protein, one aptamer or antibody binding can be used to a spike (S) protein or membrane glycoprotein while another aptamer binding to nucleocapsid protein for SARS-CoV-2 detection as an alternative (FIG. 34).

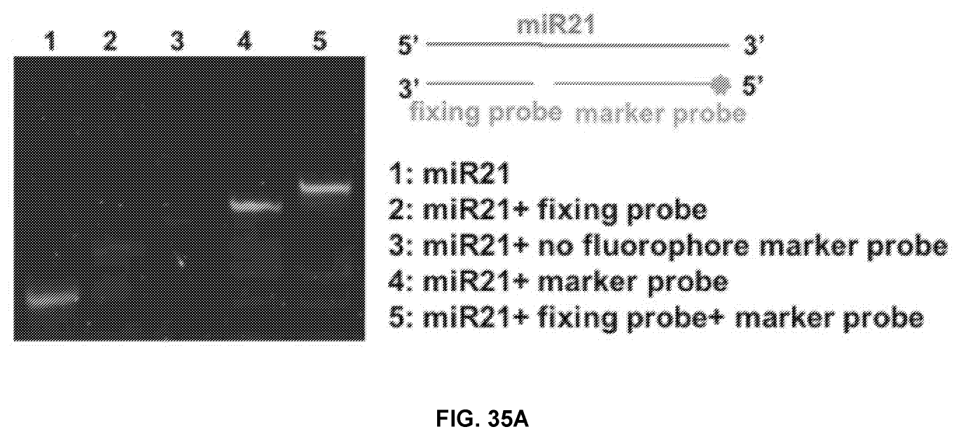

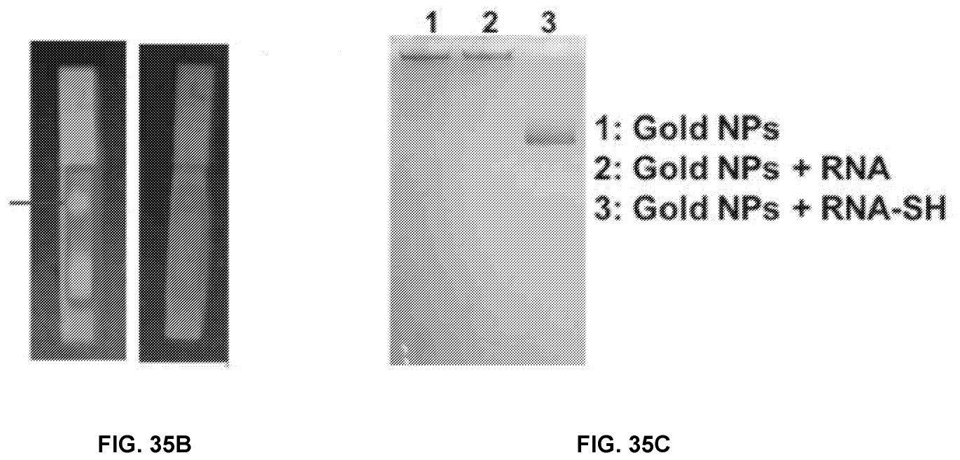

Fast oncogenic miRNA detection was actively investigated on a nitrocellulose membrane. Two RNA based probes can detect miR21 efficiently, clearly showing the positive fluorescence signal spot on nitrocellulose membrane (FIG. 35). Gold nanoparticles have also been successfully conjugated the on RNA.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A system for detecting the presence of a target molecule in a fluid sample, comprising a
a lateral flow device comprising:
a porous lateral flow test strip;
a solvent reservoir; and
an immobilization oligonucleotide that selectively binds a first portion of the target molecule affixed to the lateral flow test strip at a spot or line;
a marker oligonucleotide that selectively binds a second portion of the target molecule conjugated to a detection reagent; and
a solvent configured to drag the target molecule through the lateral flow test strip by capillary forces.

2. The system of claim 1, wherein the lateral flow test strip comprises a nitrocellulose, cellulose acetate, or glass fiber membrane.

3. The system of claim 1, wherein the target molecule is a single stranded nucleic acid molecule.

4. The system of claim 3, wherein the immobilization oligonucleotide is complementary to the first portion of the target nucleic acid, and wherein the marker oligonucleotide is complementary to the second portion of the target molecule.

5. The system of claim 3, wherein the single stranded nucleic acid molecule comprises a mRNA, ncRNA, sRNA, piRNA, miRNA, tRNA, rRNA, siRNA, lncRNA, snoRNA, snRNA, exRNA, or scaRNA.

6. The system of claim 3, wherein the single stranded nucleic acid molecule comprises Xist or HOTAIR noncoding RNA.

7. The system of claim 1, wherein the target molecule is a polypeptide.

8. The system of claim 7, wherein the immobilization oligonucleotide is a DNA aptamer, and wherein the marker oligonucleotide is a DNA aptamer.

9. The system of claim 7, wherein the target molecule comprises a viral protein.

10. The system of claim 9, wherein the viral protein comprises a SARS-CoV-2 N protein or S protein.

11. The system of claim 1, wherein the fluid sample comprises serum, plasma, urine, semen, or saliva.

12. The system of claim 1, wherein the immobilization oligonucleotide is conjugated to an immobilization agent, wherein the immobilization oligonucleotide is affixed to the lateral flow test strip by the immobilization agent.

13. The system of claim 12, wherein the immobilization agent comprises biotin.

14. The system of claim 12, wherein the immobilization agent comprises a chemical crosslinker.

15. The system of claim 12, wherein the immobilization agent comprises a lipid, wherein the lipid immobilizes the immobilization oligonucleotide to the lateral flow test strip by hydrophobic force.

16. The system of claim 1, wherein the immobilization oligonucleotide comprises one or more locked nucleic acids.

17. The system of claim 1, wherein the marker oligonucleotide comprises one or more locked nucleic acids.

18. The system of claim 1, wherein the detection agent comprises a fluorescence molecule, a dyed microsphere, or a gold nanoparticle.

19. The system of claim 1, further comprising a control oligonucleotide complementary to a portion of the marker oligonucleotide affixed to the lateral flow test strip at a control spot or line.

20. A method for detecting the presence of a target molecule in a fluid sample, comprising
(a) contacting the fluid sample with a marker that selectively binds a first portion of the target nucleic acid conjugated to a detection reagent under conditions suitable for the marker oligonucleotide to bind to the target molecule;
(b) providing a lateral flow device that comprises
a porous lateral flow test strip;
a solvent reservoir; and
an immobilization oligonucleotide that selectively binds a second portion of the target molecule affixed to the lateral flow test strip at a detection spot or line;
(c) administering the sample of step (a) to the lateral flow device of step (b) under conditions suitable for the solvent to pull the sample through the lateral flow test strip by capillary forces; and
(d) assaying the lateral flow test strip for the detection reagent at the detection spot or line.

21-38. (canceled)


SEQUENCE LISTING (8 sequences)

SEQ ID NO: 1 - RNA, 22 nt, Artificial Sequence
  Synthetic Construct
caaagugcuuacagugcaggua

SEQ ID NO: 2 - RNA, 22 nt, Artificial Sequence
  Synthetic Construct
uagcuuaucagacugauguuga

SEQ ID NO: 3 - RNA, 23 nt, Artificial Sequence
  Synthetic Construct
uuaaugcuaaucgugauaggggu

SEQ ID NO: 4 - DNA, 13 bp, Artificial Sequence
  Synthetic Construct
tcaacatcagtct

SEQ ID NO: 5 - DNA, 58 bp, Artificial Sequence
  Synthetic Construct
gctggatgtcgcttacgacaatattccttaggggcaccgctacattgacacatccagc

SEQ ID NO: 6 - DNA, 58 bp, Artificial Sequence
  Synthetic Construct
gctggatgttcatgctggcaaaattccttaggggcaccgttactttgacacatccagc

SEQ ID NO: 7 - DNA, 58 bp, Artificial Sequence
  Synthetic Construct
gctggatgttgacctttacagatcggattctgtggggcgttaaactgacacatccagc

SEQ ID NO: 8 - DNA, 58 bp, Artificial Sequence
  Synthetic Construct
gctggatgtcaccggattgtcggacatcggattgtctgagtcatatgacacatccagc